(19) United States
(12) Patent Application Publication
(10) Pub. No.: US 2025/0000834 A1
(43) Pub. Date: Jan. 2, 2025

(54) COMPOSITIONS AND METHODS FOR TARGETING GPCR FOR THE PREVENTION AND TREATMENT OF PAIN

(71) Applicant: Duke University, Durham, NC (US)

(72) Inventors: Ru-Rong Ji, Durham, NC (US); Sangsu Bang, Durham, NC (US); Sharat Chandra, Durham, NC (US)

(73) Assignee: Duke University, Durham, NC (US)

(21) Appl. No.: 18/703,125

(22) PCT Filed: Oct. 21, 2022
(86) PCT No.: PCT/US2022/047380

§ 371 Date: Apr. 19, 2024

Related U.S. Application Data
(60) Provisional application No. 63/270,198, filed on Oct. 21, 2021.

(51) Int. Cl. A61K 31/202; A61K 31/192; A61K 31/195; A61K 31/216; A61K 31/4178; A61K 31/426; A61K 31/427; A61K 31/445; A61K 31/498; A61K 31/506; A61K 31/517; A61K 31/5375; A61P 25/04

(52) U.S. Cl. CPC A61K 31/202; A61K 31/192; A61K 31/195; A61K 31/216; A61K 31/4178; A61K 31/426; A61K 31/427; A61K 31/445; A61K 31/498; A61K 31/506; A61K 31/517; A61K 31/5375; A61P 25/04

(57) ABSTRACT

Methods for treating and preventing pain are described. The methods include administering a therapeutically effective amount of a GPR37L1 ligand to a subject in need thereof. Also described herein are pharmaceutical compositions containing the GPR37L1 ligands.

### CROSS-REFERENCES TO RELATED APPLICATIONS

The present application claims priority to U.S. Provisional Pat. Appl. No. 63/270,198, filed on Oct. 21, 2021, which application is incorporated herein by reference in its entirety.

### BACKGROUND

Satellite glial cells (SGCs) wrap around neuronal cell bodies and form a complete envelope, allowing for close neuron-SGC interactions in dorsal root ganglia (DRG) and trigeminal ganglia where the cell bodies of primary sensory neurons including nociceptive neurons are present. Despite different locations in the peripheral nervous system (PNS) and central nervous system (CNS), SGCs share many features with astrocytes. For example, they express similar markers such as GFAP, GLAST, ALDH1L1, and Hevin/SPARCL1, and are interconnected by gap-junction. Like astrocytes, SGCs express high levels of inwardly-rectifying K+ channels 4.1 (Kir4.1) channels, which enables SGCs to control the perineural potassium homeostasis and neuronal excitability. Several lines of evidence indicate that SGCs participate in the generation and maintenance of chronic pain. First, the gap-junction coupling between SGCs surrounding individual neurons is augmented in pathological pain conditions such as nerve injury and inflammation. Second, Kir4.1 is downregulated under pathological pain conditions. Third, silencing Kir4.1 expression in SGCs is sufficient to induce pain hypersensitivity. Finally, upon activation, SGCs release pro-inflammatory cytokines, such as TNF-α and IL-1β that can drive hyper-excitability of surrounding sensory neurons. However, the beneficial role of SGCs in the resolution of pain has not been investigated.

G-protein coupled receptor 37-like 1 (GPR37L1) is an orphan G-protein-coupled receptor (GPCR). An early study showed that GPR37L1 and its family member GPR37 are potential receptors for the neuroprotective and glioprotective factors prosaptide and prosaposin, and furthermore, prosaptide (TX14) was shown to inhibit neuropathic pain. GPR37L1 is highly expressed in the brain and has protective role in astrocytes. Notably, GPR37L1 has been implicated in neurological diseases. GPR37L1 deletion leads to precocious cerebellar development and hypertension and increased seizure susceptibility. GPR37L1 variant in humans is associated with progressive myoclonus epilepsies (PMEs), disorders characterized by myoclonic and generalized seizures with progressive neurological deterioration. A recent study suggested that the orphan receptor GPR37L1 remains unliganded, as GPR37L1 may have constitute activities, which can be modulated by protease cleavage. Single-cell analysis revealed that the GPR37L1 transcript is highly enriched in SGCs of mouse DRG. However, the role of GRP37L1 in the PNS has not been investigated.

### BRIEF SUMMARY

Provided herein are methods for treatment of pain. The methods include administering a therapeutically effective amount of a GPR37L1 ligand to the subject in need thereof. The GPR 37L1 ligand may be a pro-resolving lipid mediator.

Exemplary embodiments provided in accordance with the present disclosed subject matter include, but are not limited to, the claims set forth herein and the following embodiments.

Some embodiments of the present disclosure provide methods for treating and/or preventing pain in a subject, the method comprising administering a therapeutically effective amount of a GPR37L1 ligand to the subject. In some embodiments, the GPR37L1 ligand is selected from the group consisting of pro-resolving lipid mediator maresin 1 (MaR1), NPD1, MCULE-6965498156, MCULE-3117827100, MCULE-5609766020, MCULE-2884027413, MCULE-1786116136, MCULE-2175470034, MCULE-9605880790, MCULE-270239856, MCULE-1615794560, MCULE-1784164690, MCULE-7360829293, MCULE-2030135661, MCULE-2500192016. MCULE-2227574192, MCULE-5725483684, MCULE-8222044844, any fragments, derivatives, salts, esters, and variants thereof, and any combinations thereof.

In some embodiments, the GPR37L1 ligand has the structure of:

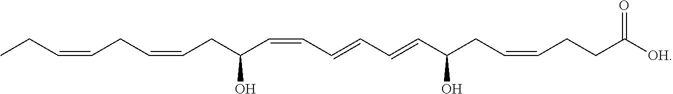 [SMILES: CC/C=C\C/C=C\C[C@H](O)\C=C/C=C/C=C/[C@H](O)C/C=C\CCC(=O)O]

In some embodiments, the GPR37L1 ligand has the structure of:

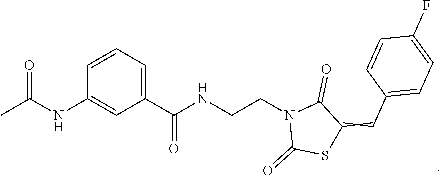

In some embodiments, the pain comprises an inflammatory pain. In another embodiment, the pain comprises neuropathic pain. In another embodiment, the pain comprises mechanical allodynia. In other embodiments, the pain comprises cancer pain. In some embodiments, the pain is at least one of headache or low-back pain.

In some embodiments, the subject is undergoing treatment for cancer. In some embodiments, the subject is undergoing a chemotherapeutic regimen or has previously been treated with a chemotherapeutic regimen. In some embodiments, the chemotherapeutic regimen comprises paclitaxel or oxaliplatin. In some embodiments, the subject is being administered radiation therapy or has previously been treated with radiation therapy. In some embodiments, the subject is a rodent (e.g., a mouse), wherein the mouse develops diabetes-induced peripheral neuropathy by administration of streptozotocin.

In some embodiments, the GPR37L1 ligand may be administered intrathetically or intraperitoneally.

In yet other embodiments, the present disclosure may relate to a pharmaceutical composition comprising a GPR37L1 ligand and a pharmaceutically acceptable diluent, excipient, and/or carrier. In some embodiments, the GPR37L1 ligand is selected from the group consisting of pro-resolving lipid mediator maresin 1 (MaR1), NPD1, MCULE-6965498156, MCULE-3117827100, MCULE-5609766020, MCULE-2884027413, MCULE-1786116136, MCULE-2175470034, MCULE-9605880790, MCULE-270239856, MCULE-1615794560, MCULE-1784164690, MCULE-7360829293, MCULE-2030135661, MCULE-2500192016. MCULE-2227574192, MCULE-5725483684, MCULE-8222044844, any fragments, derivatives, salts, esters, and variants thereof and combinations thereof.

### BRIEF DESCRIPTION OF THE DRAWINGS

FIG. 1 shows quantification of GPR37L1-positive cells in mouse DRG according to certain aspects of this disclosure. The bar graphs show quantification of GPR37L1-positive cells in DRG. n=5 mice per group. ***p<0.001, **p<0.01, unpaired t-test; FABP7 and GLAST were used as markers to isolate SGCs from DRG. Data are expressed as mean±s.e.m.

FIG. 2 is a Western blot comparing GPR37L1 expression in the brain, spinal cord, DRG, TG, sciatic nerve, kidney, heart, and spleen.

FIG. 3 is a Western blot of mouse DRG plasma membrane (PM) fraction showing that streptozotocin (STZ) treatment reduced GPR37L1 in the plasma membrane (PM) of DRGs collected 4 weeks after the injection and STZ further decreased PM expression of Kir4.1.

FIG. 4A is a Western blot of N-terminal truncated GPR37L1 expressed in DRGs of mice with diabetic neuropathy according to certain aspects of this disclosure. At the point of initial treatment with streptozotocin (STZ), which induces neuropathic pain, there is little N-terminal truncated GPR37L1 detected from the DRG sample. However, there is a time-dependent increase of N-terminal truncated GPR37L1 with continued treatment with STZ over a period of 4 weeks.

FIG. 4B is a bar graph showing ELISA quantification of soluble GPR37L1 (sGPR37L1) in urine samples of mice 0 weeks and 4 weeks after streptozotocin (STZ) treatment according to certain aspects of this disclosure. The results show a significant increase (p<0.05, t-test, n=5 mice) of soluble GPR37L1 (sGPR37L1) at 4 weeks after STZ treatment.

FIG. 5A-5B show assessment of physiological pain and neuropathic pain in Gpr37l1^{+/+}, Gpr37l1^{+/-}, and Gpr37l1^{-/-} mice according to certain aspects of this disclosure. FIG. 5A shows a bar graph pf streptozotocin (STZ)-induced mechanical allodynia in Gpr37l1^{+/+} mice (n=7), Gpr37l1^{+/-} mice (n=10), and Gpr37l1^{-/-} mice (n=8) according to certain aspects of this disclosure. Mice were treated with 75 mg/kg STZ. FIG. 5B shows bar graphs of and paclitaxel (PTX)-induced mechanical allodynia in Gpr37l1^{+/+} mice (n=7), Gpr37l1^{+/-} mice (n=10), and Gpr37l1^{-/-} mice (n=8) according to certain aspects of this disclosure. Data are expressed as the mean±SEM and were statistically analyzed by Two-Way ANOVA with Tukey's post-hoc test, * p<0.05, ** p<0.01, ****p<0.0001. Note neuropathic pain recovery is impaired in mice with Gpr37l1 deficiency.

FIG. 6 shows that a partial loss of GPR37L1 is sufficient to produce a pain state in naïve animals according to aspects of this disclosure. A knockdown experiment conducted with specific siRNA that target Gpr37l1 expression. Unilateral intraganglionic microinjections of 2 μL of Gpr37l1-targeting siRNA (4 μg) or scrambled control RNA (scRNA) in the L4 and L5 DRGs of naïve animals was performed. The siRNA reduced Gpr37l1 expression by 50% and induced persistent mechanical allodynia in naïve animals for >2 days (**P<0.01, vs. scRNA). Mechanical pain sensitivity assessed by paw withdrawal threshold (gram) in von Frey testing. The bar graph shows the data for each of baseline (BL) as well as day 1 (1d) and day 2 (2d) after injection for scRNA (left bars) and siRNA (right bars).

FIGS. 7A-7E shows that MaR1 binds GPR37L1 and induces intracellular signaling in GPR37L1-expressing cells according to certain aspects of this disclosure. FIG. 7A shows a representative blot of MaR1-coated PVDF membrane. FIG. 7B shows quantification of dot intensity in DRG lysates from WT and KO mice. *p<0.05, n=3, unpaired t-test. This data showed MaR1 binding to WT DRG lysates but not DRG lysates. FIG. 7C shows quantification of the intensity of Western blots from a lipid pull-down assay showing lipid biding to GPR37L1; Lysate and Mock are respective positive control (PC) and negative control (NC). ***p<0.001, MaR1 vs. NC, One-Way ANOVA followed by Tukey's post-hoc test. n=3 repeats. FIG. 7D is a graph with the results of a B-arrestin 2 assay in hGPR37L1 expressed Chok1 cells showing the effects of MaR1, NPD1, and TX14. Of note, MaR1 is 10 times more potent than TX14, and NPD1 is a weaker agonist, compared to MaR1. n=6 cultures from two separate experiments. FIG. 7E a graph with the results of a cAMP-BRET assay showing inhibition of forskolin-induced cAMP by MaR1 (10 nM) but not DHA (100 nM). HEK293 cells were co-transfected GPR37L1 and cAMP BRET cDNAs. ***p<0.001, n=6 from 3 experiments; One-Way ANOVA followed by Tukey's post-hoc test. Data are expressed as mean±s.e.m. The control data tracked with the DHA data.

FIGS. 8A-8C are ribbon diagrams showing docking simulations of GPR37L1 binding with MaR1 and NPD1 according to aspects of this disclosure. FIG. 8A shows the overall structure of hGRP37L1 in complex with MaR1, while FIG. 8B shows a close up view. FIG. 8C shows hGRP37L1 in complex with NPD1.

FIGS. 8D-8E are RMSD graphs showing 1000 ns simulations of GPR37L1-MaR1 complex and GPR37L1-NPD1 complex, respectively, according to certain aspects of this disclosure.

FIG. 8F is a RMSD graph showing the cluster-1 ensemble structure of GPR37L1-MaR1 simulated for 100 ns according to certain aspects of this disclosure.

FIGS. 9A-9B show that MaR1 reduces chemotherapy or diabetes-induced mechanical allodynia, respectively, in mice via GPR37L1 according to certain aspects of this disclosure. FIG. 9A shows paw withdrawal thresholds in Gpr37l1^{+/+} mice (left, n=7), Gpr37l1^{+/-} mice (middle, n=10), and Gpr37l1^{-/-} mice (n=8) after intrathecal MaR1 (100 ng) injection at 3 days after PTX injection. **** p<0.0001, ** p<0.01; ns, not significant, paired t-test. FIG. 9B shows paw withdrawal thresholds in Gpr37l1^{+/+} mice (left, n=5), Gpr37l1^{+/-} mice (middle, n=7), and Gpr37l1^{-/-} mice (n=6) after intrathecal MaR1 (100 ng) injection at 3 days after STZ injection. *** p<0.001, * p<0.05; ns, not significant, paired t-test. Abbreviations: PTX, paclitaxel; STZ, streptozotocin

FIG. 10 shows MaR1 inhibits paclitaxel-induced IL-1β release in SGC-neuron co-cultures via GPR37L1 according to certain aspects of this disclosure. IL-1β release in neuron-glia cultures from DRG of WT and Gpr37l1^{-/-} mice were analyzed by ELISA. The cultures were stimulated with 1 μM paclitaxel for 24 h in the absence or presence of MaR1 (100 nM). Note MaR1 can inhibit chemotherapy-induced IL-1β release in WT but not KO mice. Data are expressed as mean±s.e.m. *** p<0.001, * p<0.05; ns, Two-Way ANOVA, followed by Tukey's post-hoc tests. n=6 mice for WT and n=4 mice for KO mice.

FIG. 11A-11B shows MaR1 increases potassium currents in SGCs of PTX-treated DRG via GPR37L1 according to certain aspects of this disclosure. FIG. 11A part A shows total K⁺ currents in WT-DRG treated with vehicle or PTX (1 μM, 1 h). FIG. 11A part B shows total K⁺ currents in SGCs of Gpr37l1^{+/+} DRG treated with PTX (1 μM, 1 h) or PTX+MaR1 (100 ng/ml). FIG. 11A part C shows total K⁺ currents in SGCs of Gpr37l1^{+/-} DRG treated with PTX (1 μM, 1 h) or PTX+MaR1 (100 ng/ml). FIG. 11B shows amplitude of Ik-160 (B-D) with the holding potential of −160 mV. Note that the K+ currents are suppressed by PTX and MaR1 can increase the currents in WT mice but not in mutant mice. Data are expressed as mean±SEM and analyzed by One-Way ANOVA with Bonferroni's post-hoc test ** p<0.01, **** p<0.0001, n.s., not significant.

### DETAILED DESCRIPTION

The present disclosure is based, in part, on the findings by the inventors on the role of GPR37L1 in the resolution of pain. These findings relate to the expression of GPR37L1 by satellite glial cells (SGCs), the role of the Gpr37l1 gene in the resolution of chemotherapy-induced neuropathic pain, possible ligands of GPR37L1, and the effect of identified GPR37L1 ligands on potassium currents in Gpr37l1 wild-type and mutant mice.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### I. Definitions

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition (e.g., pain) manifested by a patient or to which a patient may be susceptible. The aim of treatment includes the alleviation or prevention of symptoms, slowing or stopping the progression or worsening of a disease, disorder, or condition and/or the remission of the disease, disorder or condition.

An aspect of treatment includes amelioration of a subject's symptoms, which includes slowing or stopping the progression or worsening of a disease, disorder, or condition and/or the remission of the disease, disorder or condition. As used herein, the term “ameliorate” refers to the ability to make better, or more tolerable, or reduce, a disease, condition, or disorder, and may encompass “limiting progression,” which refers to the lessening or limiting of the scope or severity of the disease or disorder or condition.

As used herein, the terms “prevent,” “preventing,” “prevention,” “prophylactic treatment” and the like refer to reducing the probability of developing a disease, disorder or condition (e.g., pain) in a subject, who does not have, but is at risk of or susceptible to developing a disease, disorder or condition.

The term “effective amount” or “therapeutically effective amount” refers to an amount sufficient to effect beneficial or desirable biological and/or clinical results.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route. By “parenteral” is meant intravenous, subcutaneous or intramuscular administration. In the methods of the subject disclosure, the compounds and/or compositions of the present disclosure may be administered alone, simultaneously with one or more other agents, or the compounds and/or compositions may be administered sequentially, in either order.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. A biological sample may be obtained directly from a subject (e.g., by blood or tissue sampling) or from a third party (e.g., received from an intermediary, such as a healthcare provider or lab technician).

The term “disease” or “condition” as used herein includes, but is not limited to, any abnormal condition and/or disorder of a structure or a function that affects a part of an organism. The disease may be caused by an external factor, such as an infectious disease, drug regime (e.g., chemotherapeutic regimen) or by internal dysfunctions, such as cancer, cancer metastasis, and the like.

As used herein, the term “pain” refers to the basic bodily sensation induced by a noxious stimulus, received by naked nerve endings, characterized by physical discomfort (e.g., pricking, throbbing, aching, etc.) and typically leading to an evasive action by the individual. Examples of pain include, but are not limited to, acute pain, chronic pain, nociceptive pain, visceral pain, somatic pain, neuropathic pain, “other” pain (e.g., dynamic and/or mechanical allodynia), and the like. As used herein, the term pain also includes chronic and acute neuropathic pain. The terms “neuropathic pain” or “neurogenic pain” can be used interchangeably and refer to pain that arises from direct stimulation of nervous tissue itself, central or peripheral and can persist in the absence of stimulus. The sensations that characterize neuropathic pain vary and are often multiple and include burning, gnawing, aching, and shooting. (See, e.g., Rooper and Brown, (2005) Adams and Victor's Principles of Neurology, 8.sup.th ed., NY, McGraw-Hill). These damaged nerve fibers send incorrect signals to other pain centers. The impact of nerve fiber injury includes a change in nerve function both at the site of injury and areas around the injury. Chronic neuropathic pain often seems to have no obvious cause, however, some common causes may include, but are not limited to, alcoholism, amputation, back, leg and hip problems, chemotherapy, diabetes, facial nerve problems, HIV infection or AIDS, multiple sclerosis, shingles, and spine surgery. For example, one example of neuropathic pain is phantom limb syndrome, which occurs when an arm or leg has been removed because of illness or injury, but the brain still gets pain messages from the nerves that originally carried impulses from the missing limb.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient). The compounds and compositions provided herein may be used in medical (i.e., used to treat a human subject) and veterinary (i.e., used to treat non-human subjects) settings. In some embodiments, the subject is a human subject suffering from pain.

As used herein, the term “salt” refers to acid or base salts of the compounds set forth herein. Illustrative examples of pharmaceutically acceptable salts are mineral acid salts (salts of hydrochloric acid, hydrobromic acid, phosphoric acid, or the like), organic acid salts (salts of acetic acid, propionic acid, glutamic acid, citric acid, fumaric acid, or the like) salts, and quaternary ammonium salts (salts formed via reaction with methyl iodide, ethyl iodide, or the like). It is understood that the pharmaceutically acceptable salts are non-toxic. Pharmaceutically acceptable salts of the acidic compounds of the present disclosure are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts. The neutral forms of the compounds can be regenerated by contacting the salt with a base or acid, and optionally isolating the parent compound in the conventional manner. The parent form of the compound may differ from the various salt forms in certain physical properties, such as solubility in polar solvents, but otherwise the salts are equivalent to the parent form of the compound for the purposes of the present disclosure.

As used herein, the term “excipient” refers to a substance that aids the administration of an active agent to a subject. By “pharmaceutically acceptable,” it is meant that the excipient is compatible with the other ingredients of the formulation and is not deleterious to the recipient thereof. Pharmaceutical excipients useful in the present disclosure include, but are not limited to, binders, fillers, disintegrants, lubricants, glidants, coatings, sweeteners, flavors and colors.

As used herein, the term “specialized pro-resolving mediator” or “SPM” are lipid mediators that are part of a larger family of pro-resolving molecules, which includes proteins and gases, that together restrain inflammation and resolve the infection. These immunoresolvents are distinct from immunosuppressive molecules as they not only dampen inflammation but also promote host defense. Exemplary SPMs are protectins, resolvins, and lipoxins.

Unless otherwise defined, all technical terms used herein have the same meaning as commonly understood by one of ordinary skill in the art to which this disclosure belongs.

### II. Methods

Resolution of acute inflammation appears to be an active process, involving the production of specialized pro-resolving mediators (SPMs), such as resolvins, protectins, and maresisns, biosynthesized from omega-3 unsaturated fatty acids. SPMs may further attenuate inflammatory pain and neuropathic pain at doses that are much lower than morphine, without producing any side effects of opioids. The SPM receptors are known to be G-protein-coupled receptors (GPCRs). Interestingly, GPCR 37-like 1 (GPR37L1) is an orphan GPCR, but its role in pain regulation is unknown. Accumulating evidence suggests an important role of satellite glial cells (SGCs) in the pathogenesis of pain.

The present disclosure provides insight in the role of GPR37L1 in the resolution of pain. In particular, the present disclosure demonstrates that GPR37L1 signaling contributes to neuropathic pain and indicates that maresin (MaR1) may regulate the resolution of pain through GPR37L1 and potassium channels.

The compounds, salts, solvates, hydrates, prodrugs, and derivatives thereof as well as any pharmaceutical compositions thereof as described herein have many uses, including for the treatment or prevention of pain. Accordingly, an aspect of the present disclosure provides a method of treating pain in a subject, the method comprising, consisting of, or consisting essentially of administering to the subject a therapeutically effective amount of a compound or pharmaceutical composition as provided herein such that the pain is treated and/or prevented in the subject.

In one aspect, provided are methods for treating and/or preventing pain in a subject, the method comprising administering a therapeutically effective amount of a GPR37L1 ligand to the subject. In some embodiments, the GPR37L1 ligand is selected from the group consisting of pro-resolving lipid mediator maresin 1 (MaR1), NPD1, MCULE-6965498156, MCULE-3117827100, MCULE-5609766020, MCULE-2884027413, MCULE-1786116136, MCULE-2175470034, MCULE-9605880790, MCULE-270239856, MCULE-1615794560, MCULE-1784164690, MCULE-7360829293, MCULE-2030135661, MCULE-2500192016. MCULE-2227574192, MCULE-5725483684, MCULE-8222044844, any fragments, derivatives, salts, esters, and variants thereof, and any combinations thereof. In some embodiments, the GPR37L1 ligand is one or more of maresin 1 (MaR1), NPD1, MCULE-6965498156, MCULE-3117827100, MCULE-5609766020, MCULE-2884027413, MCULE-1786116136, MCULE-2175470034, MCULE-9605880790, MCULE-270239856, MCULE-1615794560, MCULE-1784164690, MCULE-7360829293, MCULE-2030135661, MCULE-2500192016. MCULE-2227574192, MCULE-5725483684, or MCULE-8222044844, or any fragments, derivatives, salts, esters, or variants thereof.

In some embodiments, the method of treating and/or preventing pain in a subject comprises administering pro-resolving lipid mediator maresin 1 (MaR1), or a fragment, derivative, salt, ester, or variant thereof to the subject. MaR1 is a member of the specialized pro-resolving mediator (SPM) family of bioactive lipids.

In some embodiments, the GPR37L1 ligand may be administered intrathecally or intraperitoneally. In yet other embodiments, the GPR37L1 ligand may be administered via oral sublingual, buccal, transnasal, transdermal, rectal, intramuscular, intravenous, intraventricular, intrathecal, intraperitoneal, and subcutaneous routes.

In some embodiments, the GPR37L1 ligand is selected from the group consisting of the compounds set forth in Table 1. In some embodiments, the GPR37L1 ligand is one or more of compounds set forth in Table 1, or any fragments, derivatives, salts, esters, or variants thereof.

In some embodiments, the GPR37L1 ligand has the structure of.

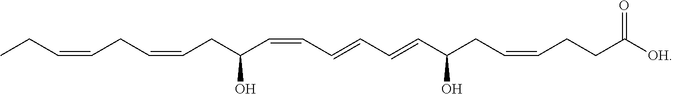 [SMILES: CC/C=C\C/C=C\C[C@H](O)\C=C/C=C/C=C/[C@H](O)C/C=C\CCC(=O)O]

In some embodiments, the GPR37L1 ligand has the structure of.

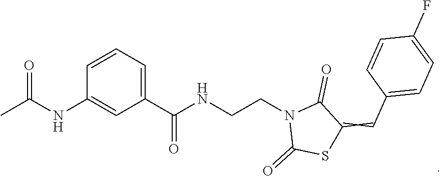

In some embodiments, the pain comprises inflammatory pain (e.g., pain as the result of the inflammation, e.g., inflammatory hyperalgesia)). In another embodiment, the pain comprises neuropathic pain such as, for example, neuropathic pain after diabetic neuropathy, chemotherapy, or traumatic brain injury. In yet other embodiments, the neuropathic pain comprises mechanical allodynia (i.e., painful sensation caused by innocuous stimuli). In other embodiments, the pain comprises cancer pain (e.g., pain as a result of a tumor pressing on nerve, bone, the spinal cord, an organ, or other tissue or as a result of weakening of bone). In other embodiments, the pain comprises headaches. In other embodiments, the pain comprises low-back pain. In some embodiments, the subject is experiencing more than one type of pain.

In some embodiments, the subject is undergoing treatment for cancer. Thus, the subject can be having administration of one or more anti-cancer agents such as a chemotherapeutic agent, including but not limited to alkylating agents, plant alkaloids, antimetabolites, anthracyclines, topoisomerase inhibitors and corticosteroids (e.g., carboplatin, paclitaxel, pemetrexed, or the like), a tyrosine kinase inhibitor (e.g., bosutinib, crizotinib, dasatinib, erlotinib, gefitinib, lapatinib, pazopanib, ruxolitinib, sunitinib, and vemurafenib, or the like), and/or an immunotherapeutic agent (e.g., pembrolizumab, nivolumab, durvalumab, atezolizumab, or the like). One or more anti-cancer agents may be administered to a subject prior to administration of the GPR37L1 ligand, concomitantly with administration of the GPR37L1 ligand, or after administration of the GPR37L1 ligand. Anti-cancer agents may be co-formulated with the GPR37L1 ligand in pharmaceutical compositions such as those as described above. In some embodiments, the subject is being treated for cancer by administration of a lactate dehydrogenase (LDH) inhibitor, a hexokinase (HK) inhibitor, or a combination thereof.

In some embodiments, the subject is undergoing a chemotherapeutic regimen in which a chemotherapeutic agent is administered to the subject. In some embodiments, the chemotherapeutic regimen comprises paclitaxel or oxaliplatin. The term “chemotherapeutic agent” refers to a compound or pharmaceutical composition useful for treating or ameliorating cancer. The agent can be given with a curative intent, with an aim to prolong life, or for the purpose of reducing symptoms. Chemotherapeutic agents include, but are not limited to, aldesleukin, alectinib anaplastic lymphoma kinase, cabozantinib, elotuzumab, fluoxymesterone, iobenguane, imiquimod, interferon, ixazomib, lanreotide, lentinan, mitotane, nab-paclitaxel, necitumumab, octreotide, somatostatin, omacetaxine, sipuleucel-T, tegafur/gimeracil/oteracil and tegafur/uracil. In some embodiments, where the subject is a rodent (e.g., a mouse), the chemotherapeutic regimen comprises streptozotocin, wherein the mouse develops diabetes-induced peripheral neuropathy by administration of streptozotocin.

Additional chemotherapeutic agents include, but are not limited to, 14regelatini, capecitabine, carmofur, cladribine, clofarabine, cytarabine, decitabine, floxuridine, fludarabine, fluorouracil, gemcitabine, mercaptopurine, nelarabine, pentostatin, tegafur, tioguanine, trifluridine/tipiracil, methotrexate, pemetrexed, pralatrexate, raltitrexed, hydroxycarbamide, irinotecan, topotecan, daunorubicin, doxorubicin, epirubicin, idarubicin, mitoxantrone, valrubicin, etoposide, teniposide, cabazitaxel, docetaxel, paclitaxel, vinblastine, vincristine, vindesine, vinflunine, vinorelbine, bendamustine, busulfan, carmustine, chlorambucil, chlormethine, cyclophosphamide, dacarbazine, fotemustine, ifosfamide, lomustine, melphalan, streptozotocin, temozolomide, trabectedin, carboplatin, cisplatin, nedaplatin, oxaliplatin, altretamine, bleomycin, bortezomib, carfilzomib, dactinomycin, eribulin, estramustine, ixabepilone, mitomycin, procarbazine, abarelix, abiraterone, anastrozole, bicalutamide, cyproterone, degarelix, enzalutamide, exemestane, flutamide, fulvestrant, goserelin, histrelin, letrozole, leuprolide, mifepristone, nilutamide, tamoxifen, toremifene, triptorelin, ibritumomab tiuxetan, radium Ra 223 dichloride, strontium-89, samarium (153Sm) lexidronam, tositumomab, ado-trastuzumab emtansine, alemtuzumab, bevacizumab, blinatumomab, brentuximab vedotin, cetuximab, daratumumab, denosumab, dinutuximab, gemtuzumab ozogamicin, ibritumomab tiuxetan, ipilimumab, nivolumab, 15regelatiniz, ofatumumab, panitumumab, pembrolizumab, pertuzumab, ramucirumab, rituximab, tositumomab, trastuzumab, afatinib, aflibercept, axitinib, bosutinib, cobimetinib, crizotinib, dasatinib, erlotinib, gefitinib, imatinibl, lapatinibl, lenvatinibl, nilotinib, 15regelatini, pazopanib, ponatinib, regorafenib, ruxolitinib, sorafenib, sunitinib, trametinib, vandetanib, everolimus, temsirolimus, alitretinoin, bexarotene, isotretinoin, tamibarotene, tretinoin, lenalidomide, pomalidomide, thalidomide, belinostat, 15regelatiniz, romidepsin, valproate, vorinostat, anagrelide, arsenic trioxide, asparaginase, Bacillus Calmete-Guerin vaccine, ceritinib, dabrafenib, denileukin diftitox, idelalisib, ibrutinib, 15regelat, 15regelatini, sonidegib, talimogene laherparepvec, vemurafenib, and vismodegib. The chemotherapeutic agents may also include the salts, hydrates, solvates and prodrug forms of any of the above referenced chemotherapeutic agents.

In some embodiments, the subject is being treated for cancer with administration of radiotherapy, e.g., external beam radiation; intensity modulated radiation therapy (IMRT); brachytherapy (internal or implant radiation therapy); stereotactic body radiation therapy (SBRT)/stereotactic ablative radiotherapy (SABR); stereotactic radiosurgery (SRS); or a combination of such techniques.

In some embodiments, the subject has previously been treated for cancer with a chemotherapeutic agent and/or radiation therapy. In some embodiments, the subject has been treated for cancer by surgical removal or ablation of a tumor or cancerous cells.

In some embodiments, the subject is a rodent (e.g., a mouse), wherein the mouse develops diabetes-induced peripheral neuropathy by administration of streptozotocin.

### III. Compositions

Some embodiments of the present disclosure may relate to a pharmaceutical composition comprising a GPR37L1 ligand and a pharmaceutically acceptable diluent, excipient, and/or carrier. In some embodiments, the GPR37L1 ligand is selected from the group consisting of pro-resolving lipid mediator maresin 1 (MaR1), NPD1, MCULE-6965498156, MCULE-3117827100, MCULE-5609766020, MCULE-2884027413, MCULE-1786116136, MCULE-2175470034, MCULE-9605880790, MCULE-270239856, MCULE-1615794560, MCULE-1784164690, MCULE-7360829293, MCULE-2030135661, MCULE-2500192016. MCULE-2227574192, MCULE-5725483684, MCULE-8222044844, any fragments, derivatives, salts, esters, and variants thereof and combinations thereof. In some embodiments, the GPR37L1 ligand is one or more of maresin 1 (MaR1), NPD1, MCULE-6965498156, MCULE-3117827100, MCULE-5609766020, MCULE-2884027413, MCULE-1786116136, MCULE-2175470034, MCULE-9605880790, MCULE-270239856, MCULE-1615794560, MCULE-1784164690, MCULE-7360829293, MCULE-2030135661, MCULE-2500192016. MCULE-2227574192, MCULE-5725483684, or MCULE-8222044844, or any fragments, derivatives, salts, esters, or variants thereof.

Pharmaceutical compositions comprising the GPR37L1 ligands may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The GPR37L1 ligands may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, but not limited to, for example, topical, ocular, oral, buccal, systemic, nasal, injection, transdermal, rectal, vaginal, intrathecal, intra-articular, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal, peri-neural, or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, PEG-40 hydrogenated castor oil (e.g., Cremophore™) or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt.

Alternatively, transdermal delivery systems manufactured as an adhesive disc or patch which slowly releases the compound(s) for percutaneous absorption may be used. To this end, permeation enhancers may be used to facilitate transdermal penetration of the GPR37L1 ligands(s).

Alternatively, other pharmaceutical delivery systems may be employed. Liposomes and emulsions are well-known examples of delivery vehicles that may be used to deliver the GPR37L1 ligands(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the GPR37L1 ligands(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of the GPR37L1 ligands(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular GPR37L1 ligands(s) the conversation rate and efficiency into active drug compound under the selected route of administration, etc.

Determination of an effective dosage of the GPR37L1 ligands(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC50 of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. The dose of the GPR37L1 ligand(s) can be, for example, about 0.01-750 mg/kg, or about 0.01-500 mg/kg, or about 0.01-250 mg/kg, or about 0.01-100 mg/kg, or about 0.1-50 mg/kg, or about 1-25 mg/kg, or about 1-10 mg/kg, or about 5-10 mg/kg, or about 1-5 mg/kg. The dose of the GPR37L1 ligand(s) can be about 0.01, 0.05, 0.1, 0.5, 1, 5, 10, 15, 20, 25, 30, 35, 40, 45, 50, 55, 60, 65, 70, 75, 80, 85, 90, 95, or 100 mg/kg.

Dosage amount and interval may be adjusted individually to provide plasma levels of the GPR37L1 ligand(s) which are sufficient to maintain therapeutic or prophylactic effect. For example, the compound(s) may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

It will be appreciated that the actual preferred method and order of administration will vary according to, inter alia, the particular preparation of interfering molecules being utilized, the particular formulation(s) of the one or more other interfering molecules being utilized. The optimal method and order of administration of the compounds and/or compositions of the disclosure for a given set of conditions can be ascertained by those skilled in the art using conventional techniques and in view of the information set out herein. In accordance with good clinical practice, it is preferred to administer the instant compounds and/or compositions at a concentration level which will produce effective beneficial effects without causing any harmful or untoward side effects.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### EXAMPLES

The following Examples are provided by way of illustration and not by way of limitation.

### Example 1: Materials and Methods

The following materials and methods were used to conduct the experiments described in Examples 2-8 of this disclosure.

Reagents: This Examples of this disclosure used the following reagents and concentrations: Maresin 1 (MaR1), NPD1/PD1, RvD1, RvD2, RvD3, and DHA were purchased from Cayman Chemical Company (Ann Arbor, MI). TX14 peptide was purchased from AnaSpec (Fremont, CA). Nano-lantern (cAMP-1.6)/pcDNA3 (Addgene plasmid #53594; RRID: Addgene_53594 (Watertown, MA)). GPR37L1-Tango, were from Addgene: Plasmid #66356; RRID: Addgene_66356 (Watertown, MA)).

Animals: All the mouse procedures were approved by the Institutional Animal Care & Use Committee of Duke University (IACUC). Animal experiments were conducted according to the National Institutes of Health Guide for the Care and Use of Laboratory Animals. B6; 129S5-Gpr37l1^{tmlLex}/Mmucd strain was obtained from UC Davis (MMRRC, stock #011709-UCD). Gpr37l1 and littermate mice were maintained at Duke University Medical Center. Adult mice (males and females, 8-10 weeks) were used for behavioral tests and biochemical assays. Two to five mice were housed in each cage under a 12-hour alternating light-dark cycle with ad libitum access to food and water.

Pain models and drug injection: Animal model of chemotherapy-induced peripheral neuropathy (CIPN) was induced by paclitaxel (Sigma-Aldrich (St. Louis, MO), T7191, diluted with sterile saline). A single injection of paclitaxel (6 mg/ml) was given by intraperitoneal injection. A single injection of paclitaxel may induce mechanical allodynia for 4-6 weeks (X. Luo et al., Macrophage Toll-like Receptor 9 Contributes to Chemotherapy-Induced Neuropathic Pain in Male Mice. J Neurosci 39, 6848-6864 (2019)). Animal model of dietetic neuropathy was induced by streptozotocin (STZ, Sigma-Aldrich (St. Louis, MO), S0130, diluted with sterile saline). A single injection of STZ (75 mg/kg) was given as previously described (N. C. Brigham et al., Controlled release of etoricoxib from poly(ester urea) films for post-operative pain management. J Control Release 329, 316-327 (2021)). For intrathecal (i.t.) injection of MaR1, a spinal cord puncture was made by a Hamilton micro-syringe (Hamilton, Reno, NV) with a 30-gauge needle between the L5 and L6 levels to deliver reagents (5 μl) to the cerebral spinal fluid (J. L. Hylden, G. L. Wilcox, Intrathecal morphine in mice: a new technique. Eur. J Pharmacol. 67, 313-316 (1980)).

HEK293 cell culture and GRP37L1 transfection: The HEK293 Flp-In™ cell line (Invitrogen (Waltham, MA), R78007) was purchased from the Duke Cell Culture Facility. The cells were cultured in high glucose (4.5 g/L) Dulbecco's Modified Eagle's Medium containing 10% (v/v) fetal bovine serum (Gibco, Waltham, MA). The GPR37L1 cDNA was transfected using Lonza 4D-Nucleofector™×unit (2 μg cDNA/1×10⁷ cells; protocol #CM130). When cells reached 70% confluency, the transfected cells were further cultured for 48 h before use.

SGC and neuron co-culture: DRG were collected from wild-type (WT) or knock-out (KO) mice (8 weeks male) and incubated in 1 mg/ml Collagenase/Dispase (Roche Diagnostics, Madison, WI, USA) at 37° C. for 60 min, with agitation at 100 revolutions per minute (RPM) and then followed by incubation in 0.05% trypsin/EDTA for 10 min. The digestion enzymes were prepared in Dulbecco's modified Eagle medium/F12 with GlutaMAX (ThermoFisher, Waltham, MA). After incubation of 0.1% trypsin inhibitor and centrifugation (300 G), the cell pellet was gently triturated in a neurobasal medium containing 0.5 μM glutamine. Dissociated DRG cells were seeded on non-coated culture dishes for 4 hrs. SGC and neurons were included for co-culture by hand-shaking of the culture flasks gently for 5 to 10 min and then resuspended by replacing with a new neurobasal medium. The attached glial-like cells were cultured in the DMEM/F12 medium containing 10% fetal bovine serum and 1% streptomycin/penicillin to promote cell growth and inhibition of differentiation. After 2 to 6 days, the glial-like cells were differentiated by application of the serum-free neural basal medium. The neuron-rich fraction was seeded on a Poly L lysine (Sigma-Aldrich, St. Louis, MO) coated plate with Neurobasal+2% B27 (ThermoFisher, Waltham, MA) containing media (J. N. Poulsen, et al., Primary culture of trigeminal satellite glial cells: a cell-based platform to study morphology and function of peripheral glia. Int J Physiol Pathophysiol Pharmacol 6, 1-12 (2014)).

Lipids overlay assay: Lipids membrane coating and protein overlay assay were performed as previously described (S. Dowler, et al., Protein lipid overlay assay. Sci STKE 2002, p 16 (2002)). Lipid mediators (RvD1, RvD2, RvD3, NPD1, MaR1, DHA, and vehicle EtOH) were directly loaded on hydrophobic NC membranes walls (96 well plate; Bio-Rad, Hercules, CA). Compound-coated membranes were dried and blocked with 1% BSA. The coated membranes were incubated with lysates obtained from hGPR37L1-transfected HEK293 cells or mouse DRG lysate from WT or Gpr37l1 KO mice for 2 hours, followed by detection using an anti-GRP37L1 antibody (Bioss (Woburn, MA), Rabbit, 1:1000, #bs-15390R) or anti-flag antibody (Cell Signaling Technology (Danvers, MA), Rabbit, 1:1000, #14793). Blots were further incubated with an HRP-conjugated secondary anti-rabbit antibody (Jackson ImmunoResearch Laboratories (West Grove, PA), raised in donkey, 1:5000), developed in ECL solution (Pierce Biotechnology, Waltham, MA), and the signal was visualized from ChemiDoc XRS (Bio-Rad, Hercules, CA). The signal Intensity was quantified by Image J software (NIH).

Lipid pull-down assay: Isolated membrane proteins were pre-adsorbed to uncoated control agarose beads (Vector Laboratories, Burlingame, CA); the unbound fraction was collected and incubated 24 hrs at 4° C. with lipid-coated agarose beads (S. Bang et al., GPR37 regulates macrophage phagocytosis and resolution of inflammatory pain. J Clin Invest 128, 3568-3582 (2018)). After extensive washing, bound proteins were eluted from the lipid-coated beads, suspended in Laemmli buffer containing 2% SDS (w/v) and 0.3-M β-mercaptoethanol (Sigma-Aldrich, St. Louis, MO), and heated 5 min at 95° C. to dissolve proteins before separation on 4-20% polyacrylamide/SDS gels as outlined above. The lysate proteins were detected by western blot.

Western blot: GPR37L1 proteins were isolated from transfected cells, lipid pull-down beads, and mouse DRG. The cells were placed on ice, washed with ice-cold phosphate-buffered saline (PBS), and harvested with ice-cold RIPA buffer (Sigma-Aldrich, St. Louis, MO) with Protease Inhibitor Cocktail Tablet (pH 7.4) (Roche Diagnostics, Indianapolis, IN). The cell lysates were centrifugated for removing insoluble debris and were calculated by BCA assay for detection of protein level, and the supernatant was mixed with 4× Laemmli buffer (Bio-Rad, Hercules, CA) and boiling. Protein samples were electroporated on 4-15% gradient SDS-PAGE gel (Bio-Rad, Hercules, CA) and blotted on PVDF membrane (Bio-Rad, Hercules, CA). Ponceau S staining was used for the detection of total proteins. The primary antibody was incubated with PBS+1% BSA at 4° C. overnight. We used the following primary antibodies: anti-GPR37L1 antibody (Bioss (Woburn, MA), Rabbit, 1:1000, #bs-15390R); anti-GAPDH antibody (Proteintech (Rosemont, IL), mouse, 1:2000, #60004); and anti-flag antibody (Cell Signaling Technologies (Danvers, MA), Rabbit, 1:1000, #14793). Blots were further incubated with an H1RP-conjugated secondary anti-rabbit antibody (Jackson ImmunoResearch Laboratories (West Grove, PA), donkey, 1:5000), developed in ECL solution (Pierce Biotechnology, Appleton, WI)), and visualized in ChemiDoc XRS (Bio-Rad, Hercules, CA). Protein signal intensity was quantified by Image J software (NIH).

Beta-arrestin assay: To screen for GPR37L1 ligands, the PathHunter® eXpress GPR37L1 CHO-K1 kit was purchased from Eurofins Discoveryone™ (DiscoverX Solutions for Drug Discovery, Fremont, CA; Cat #93-0378E2ACP1M). CHO-K1 cells express human GPR37L1 and were grown in 96-well plates for 48 hours. Lipid compounds were incubated with the ChoK1 cells for 1 hour at 37° C., and GPR37L1 activation was determined by measuring chemiluminescence using the beta-arrestin 2 (β-arrestin) detection kit (DiscoverX Solutions for Drug Discovery, Fremont, CA). The luminescent activity was measured using an Infinite F200 Pro Illuminometer (Tecan Trading AG, Switzerland).

cAMP BRET assay: To detect cAMP change after GPCR37L1 activation, we transfected a Nano-lantern cAMP plasmid (1 μg) and human GPR37L1-tango plasmid (1 μg) into HEK-293 cells. BRET activity was measured upon the addition of 100 μl/well for a final concentration of 10 μM Coelenterazine (Santa Cruz Biotechnology, Dallas, TX), 10 nM MaR1, DHA or vehicle, and 100 μM forskolin (Enzo Life Sciences (Farmingdale, NY), #BML-CN100). The luminescent activity was measured using an Infinite F200 Pro Illuminometer (Tecan Trading AG, Switzerland). The luminescent activity was normalized to basal signal levels.

Flow cytometry: For the detection of SGCs, DRG tissues were dissociated by 1 mg/ml Collagenase/Dispase (Roche Diagnostics, Indianapolis, IN) in a shaking incubator for 90 min. The dissociated tissues were incubated in 10% FBS supplemented DMEM media at 1 hour for neutralization of the enzymes. The dissociated cells were washed out using a PBS+10 mM EDTA solution. The cells were fixed with 2% PFA and permeabilized with HBSS+2% triton X100. All dissociated cells were blocked with Fc receptors staining buffer (1% anti-mouse CD16/CD32, 2.4 G2, 2% FBS, 5% NRS, and 2% NMS in HBSS; BD Biosciences (Franklin Lakes, NJ)) and then stained with a standard panel of antibodies (Glast-PE(rat-IgG, cat #130-118-483, 1:200, Miltenyl Biotech (Auburn, CA)), Nissle-cy5 (Sigma-Aldrich (St. Louis, MO), 1 μg/ml), CD45-FITC (rat-igG, cat #11-0451-82, 1:200, eBiosciences (San Diego, CA)), FABP7-Cy3 (Mouse IgG, Cat #M022188, Neuromics (Edina, MN), 1 μg/ml) and GPR37L1-Apc-cy7, (Rabbit IgG, Cat #bs-15390R, Bioss (Woburn, MA), 1 μg/ml)). After staining, cells were washed in PBS with EDTA. The flow cytometry events were acquired in a BD FACS Canto II flow cytometer by using BD FACS Diva 8 software (BD Bioscience, Franklin Lakes, NJ). Data were analyzed using Cytobank Software (Cytobank, Santa Clara, CA).

Computer simulations: A sequence of human GPR37L1 was downloaded from the UniProt database (ID: 060883) infasta format (Universal Protein Resource (UniProt), Geneva, Switzerland). The predicted topology for GPR37L1 in UniProt was used for seven-transmembrane alignments and long loop identification. Template selection and homology modeling were performed using the automated modeling server GPCR-ModSim (M. Esguerra, et al., GPCR-ModSim: A comprehensive web based solution for modeling G-protein coupled receptors. Nucleic Acids Res 44, W455-462 (2016)). Human ETB (PDB: 6IGK) was selected as a template and active conformation of the model was generated. All other loops were also refined by the Prime module. Ligands were drawn in the Maestro suite in 2D format and were structurally preprocessed using LigPrep from the Schrodinger Suite (Schrödinger Release 2018-4: LigPrep, Schrödinger, New York, NY). Protonation at a physiological pH (7±2) and energy minimization was performed using the OPLS3 force field. To elucidate the binding mode of all ligands in the binding site of the homology model of hGPR37L1, docking studies were performed with the help of Autodock 4 software (G. M. Morris et al., AutoDock4 and AutoDockTools4: Automated docking with selective receptor flexibility. J Comput Chem 30, 2785-2791 (2009)). Before the docking, the hGPR37L1 structure was prepared using the AutoDock Tools 4 software. The stability and intra-molecular conformational changes of the protein, molecular dynamics simulations (MDS) were performed on a 100 ns time scale for the protein-ligand complex and 1000 trajectory structures were recorded. Using the GPU-accelerated DESMOND software (K. J. Bowers et al. Scalable algorithms for molecular dynamics simulations on commodity clusters. SC '06: Proceedings of the 2006 ACMIEEE Conference on Supercomputing, November 2006, pp. 84-es), doi.org/10.1145/1188455.1188544, the top-scored docking poses were subjected to solvent-explicit, all-atom MDS. The OPLS3 force field was used for model generation of the protein-ligand complex, energy minimization, and MDS. The protein was inserted into the POPC lipid bilayer and the full system was immersed in a periodic orthorhombic water box TIP3P. The NPT ensemble class was used with the temperature set to 300 K and pressure set to 1.01325 bar. The trajectory clustering method of Desmond was used to cluster 1000 trajectory structures into ten clusters based on atomic root mean square deviation (RMSD). Then 100 ns MDS was performed on the cluster 1 structure to assess the stability of the docking complex.

Chemical library virtual screening: Virtual screening workflow included a similarity search of a library of 10 million compounds from Mcule database, followed by molecular docking of the selected structures and absolute binding affinity predictions between the ligand-protein complex and molecular dynamics simulation. The preparation of Mcule database compounds was carried out by employing ligand preparation module of Schrodinger suite 2019-4 (Schrödinger, New York, NY). Using the default setting in the ligand preparation module, the three-dimensional (3D) low energy conformers were generated. Also, the ionization states were achieved in the pH range of 7.0±2.0 for all the compounds. KDEEP, a protein-ligand binding affinity predictor tool based on 3D convolutional neural networks was used to determine the absolute binding affinity of each ligand-GPR37L1 complex. The DCNNs model has been pre-trained, tested, and verified using the PDBbind v.2016 database, and the KDEEP tool is built on it. Two inputs were created, and GPR37L1 and all the compounds combined sdf files were given in order to run the KDEEP utility application. In the web application, the other input features were set to default. The final list of compounds for experimental evaluation is given in Table 1.

In situ hybridization using RNA scope probes: Mice were transcardially perfused with PBS followed by 4% paraformaldehyde under deep anesthesia with isoflurane. Lumbar dorsal root ganglia (DRG) and trigeminal ganglia (TG) were isolated and post-fixed in the same fixative. DRG and TG tissues were embedded in an OCT medium (Tissue-Tek Genie® O.C.T. Compound) and cryosectioned with 14 μm-thick DRG sections. The RNA scope probes against mouse Gpr37l1 (Cat No. 712651) and Gpr37 (Cat No. 319291) were designed by Advanced Cell Diagnostics USA (Newark, CA) and the RNAscope multiplex fluorescent assay was conducted according to manufacturer's instructions. Pre-hybridization and hybridization were performed according to standard methods (C. R. Donnelly et al., STING controls nociception via type I interferon signalling in sensory neurons. Nature 2021 March; 591(7849):275-280.).

Patch-clamp recordings in SGCs of whole-mount DRG: Under urethane anesthesia, mice were rapidly euthanized, followed by careful isolation of lumbar DRGs placed in the oxygenated artificial cerebral spinal fluid (aCSF). DRGs were briefly digested (20 min) using an enzymatic mixture consisting of collagenase A (1 mg/mL) and 0.32 mL Trypsin (0.25% original solution). Intact DRG were then incubated in aCSF oxygenated with 95% O₂ and 5% CO₂ at 34° C. Following incubation, DRG were transferred to a recording chamber. Isolated DRGs were transferred to a recording chamber continuously perfused (˜3 ml/min) with aCSF. SGCs in whole mouse DRGs could be visualized using a 40× water-immersion objective on an Olympus BX51WI microscope (Olympus Corp., Shinjuku, Japan). The round or fusiform-shaped cell bodies of SGCs were small (<10 μm) but visible near the edges of DRG neurons. Patch pipettes (Chase Scientific Glass Inc., Ramsey, MN) were pulled and filled with a pipette solution containing: 126 mM potassium gluconate, 10 mM NaCl, 1 mM MgCl₂, 10 mM EGTA, 2 mM Na-ATP, and 0.1 mM Mg-GTP, adjusted to pH 7.3 with KOH. The resistance of pipettes was 10-12 MΩ. A Whole-cell patch-clamp configuration was made on SGCs at room temperature using a Multiclamp 700B amplifier (Axon Instruments, Union City, CA). Under voltage clamp, at a holding potential of −80 mV, inward or outward currents are triggered by voltage steps of −160 to +40 mV, with 20 mV increment in 200 ms (H. Zhang et al., Altered functional properties of satellite glial cells in compressed spinal ganglia. Glia 57, 1588-1599 (2009)). To isolate inwardly rectifying potassium current (Kir), potassium currents from the same cells were recorded in the absence and presence of 100 μM extracellular barium, which blocks the Kir4.1 channel (C. A. Doupnik, et al, The inward rectifier potassium channel family. Curr Opin Neurobiol 5, 268-277 (1995); X. Tong et al., Astrocyte Kir4.1 ion channel deficits contribute to neuronal dysfunction in Huntington's disease model mice. Nat Neurosci 17, 694-703 (2014)). The Kir4.1 currents were obtained by digitally subtracting those currents in the absence and presence of barium.

Nociceptive behavior tests: Gpr37l1^{+/+}, Gpr37l1^{+/-}, or Gpr37l1^{-/-} mice were habituated to the testing environment for at least two days before the baseline testing. All the animal behaviors were tested blindly. Thermal and mechanical sensitivity was tested before and after the injection of paclitaxel (PTX, 6 mg/kg, IP) and streptozotocin (STZ, 75 mg/kg, IP). MaR1 was intrathecally injected 3 days after the STZ and PTX injection. For testing mechanical sensitivity, mice were confined in boxes (14×18×12 cm) placed on an elevated metal mesh floor and stimulated their hind paws with a series of von Frey hairs with logarithmically increasing stiffness (0.16-2.00 g, Stoelting (Wood Dale, IL)), presented perpendicularly to the central plantar surface. The 50% paw withdrawal threshold was measured by Dixon's up-down method (W. J. Dixon, Efficient analysis of experimental observations. Annu Rev Pharmacol Toxicol 20, 441-462 (1980)). Thermal sensitivity was measured using a Hargreaves radiant heat apparatus (IITC Life Science, Woodland Hills, CA) (K. Hargreaves, et al., A new and sensitive method for measuring thermal nociception in cutaneous hyperalgesia. Pain 32, 77-88 (1988)). The basal paw withdrawal latency was adjusted to 10-15 s, with a cutoff of 25 s to prevent tissue damage. Acetone (50 μL) was sprayed through wire mesh flooring onto the plantar surface of the infected hindpaw to produce evaporative cooling (X. Luo et al., Macrophage Toll-like Receptor 9 Contributes to Chemotherapy-Induced Neuropathic Pain in Male Mice. J Neurosci 39, 6848-6864 (2019)).

Statistics: All data were expressed as the mean±SEM. The sample size for each experiment was indicated in figure legends. GraphPad Prism 8.0 Software was used to perform Statistical analysis. The data were analyzed by two-way ANOVA, followed by Bonferroni's post-hoc test or Tukey's post-hoc test for multi-group comparison or Student t-test for two-group comparison. P<0.05 was considered as statistically significant. Statistical significance was indicated as: *p<0.05, ** p<0.01, ***p<0.001, ****p<0.0001.

### Example 2: Examining GPR37L1 Expression Under Normal and Chronic Pain Conditions

### A. Mouse SGCs Express Gpr37l1 and GPR37L1

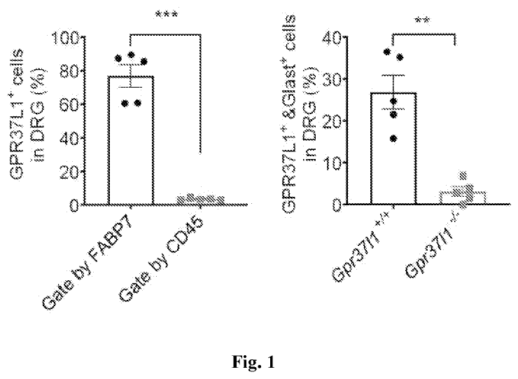

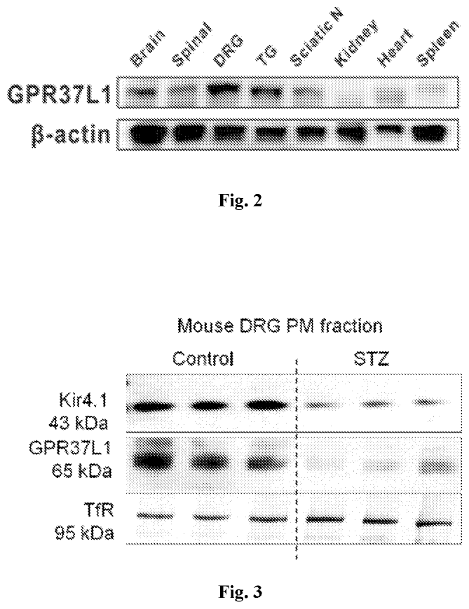

In situ hybridization with double staining using sensitive RNAscope probes revealed the distinct cellular location of Gpr37 and Gpr37l1 in mouse DRG (data not shown; see U.S. Prov. No. 63/270,198, Supplemental FIG. 1). Gpr37 was expressed by DRG neurons with large cell bodies; while Gpr37l1 was expressed in non-neuronal cells surrounding DRG neurons (data not shown; see U.S. Prov. No. 63/270,198, FIG. 1A). Not intending to be bound by theory, the unique cellular distributions suggest a distinct function of GPR37 and GPR37L1 in different cells of DRG. Further analysis with DAPI co-staining showed Gpr37l1 in SGCs but not neurons (data not shown; see U.S. Prov. No. 63/270,198, FIG. 1B). Notably, Gpr37l1 signal was completely eliminated in DRG of Gpr37l1^{-/-} (KO) mice, validating the specificity of the staining (data not shown; see U.S. Prov. No. 63/270,198, FIG. 1C). Gpr37l1^{-/-} mRNA expression was also observed in SGCs of TG neurons and nodose ganglia (NG) (data not shown; see U.S. Prov. No. 63/270,198, Supplemental FIG. 2).

Next, Western blot was used to examine GPR37L1 protein expression in mouse DRG tissues of WT and KO mice in their genotypes (Gpr37l1^{+/+}, Gpr37l1^{+/-}, and Gpr37l1^{-/-}. A GPR37L1-specific band (˜50 kDa) was observed in WT DRG; however, this specific band was reduced in heterozygote (Gpr37L1^{+/-}) mice, and completely eliminated in homozygote (Gpr37l1^{-/-}) mice (data not shown; see U.S. Prov. No. 63/270,198, FIG. 1D).

To further determine the SGC localization of GPR37L1, flow cytometry analysis was used in mouse DRG to detect the plasma membrane expression of GPR37L1 (data not shown; see U.S. Prov. No. 63/270,198, FIG. 1E). The co-expression of GPR37L1 with FABP7 or GLAST was examined as a marker for SGC in DRG of Gpr37l1^{+/+} and Gpr37L1^{-/-} mice. (See O. Avraham et al., Profiling the molecular signature of Satellite Glial Cells at the single cell level reveals high similarities between rodent and human. bioRxiv (2021); O. Avraham et al., Satellite glial cells promote regenerative growth in sensory neurons. Nat Commun 11, 4891 (2020); S. B. Jager, et al., Isolation of satellite glial cells for high-quality RNA purification. J Neurosci Methods 297, 1-8 (2018)). GPR37L1 was co-expressed in 80% of FABP7⁺ cells and 25% GLAST⁺ cells, but only in 2% of CD45⁺ cells (immune cell marker) in WT mice (FIG. 1). Notably, GPR37L1 signaling was largely abolished in Gpr37l1^{-/-} mice (p<0.01, n=5 mice/group (FIG. 1).

### B. GPR37L1 Distribution in Different Mouse Tissues

To determine the therapeutic effects and potential toxicity of GPR37L1 agonist, GPR37L1 expression in different tissues was determined. GPR37L1 expression was compared in brain, spinal cord, DRG, TG, sciatic nerve, kidney, heart, and spleen (FIG. 2). It was determined by Western blots that full length GPR37L1 (˜55 kD) is highly expressed in the PNS including DRG and TG. The GPR37L1 band was also expressed in the CNS including brain and spinal cord. The expression was very low in heart, kidney, and spleen.

### C. Altered Expression of GPR37L1 in Mouse Neuropathic Pain Conditions

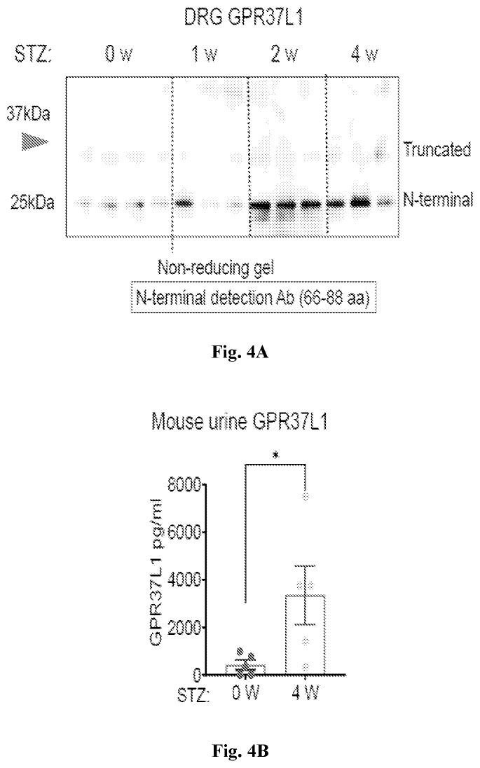

Substantial reductions of DRG surface expression of GRP37L1 were found after diabetic peripheral neuropathy (DPN). Streptozotocin (STZ) treatment not only induced neuropathic pain but also reduced GPR37L1 in the plasma membrane (PM) of DRGs collected 4 weeks after the injection (FIG. 3). STZ further decreased PM expression of Kir4.1 (FIG. 3). In contrast, time-dependent increase of N-terminal truncated GPR37L1 in DRGs of diabetic mice was observed (FIG. 4A). Truncated GPR37L1 can be secreted into circulation after the full-length protein is cleaved by certain enzyme. ELISA analysis also detected a significant increase (p<0.05, t-test, n=5 mice) of soluble GPR37L1 (sGPR37L1) in urine samples in mice 4 weeks after the STZ treatment (FIG. 4B). Not intending to be bound by theory, these results suggest that full-length GPR37L1 in DRGs could be cleaved and released to urine under diabetic neuropathy condition.

### D. GPR37L1-Expression in Human DRG Tissues and the Impact of Chemotherapy

GPR37L1 expression was examined in human DRG tissues of non-diseased donors from National Disease Research Interchange (NDRI) (see W. Chang et al. 2018, Expression and Role of Voltage-Gated Sodium Channels in Human Dorsal Root Ganglion Neurons with Special Focus on Nav1.7, Species Differences, and Regulation by Paclitaxel. Neurosci Bull 34(1): 4-12; Z. Z. Xu et al. 2015 Inhibition of mechanical allodynia in neuropathic pain by TLR5-mediated A-fiber blockade. Nat Med 21(11):1326-1331). Profiling the molecular signature of SGCs at the single cell level revealed high similarities between rodent and human (see L. Yang et al., Human and mouse trigeminal ganglia cell atlas implicates multiple cell types in migraine. Neuron 110(11):P1806-1821.E8 (Epub Mar. 28, 2022); O. Avraham, et al., 2022, Profiling the molecular signature of satellite glial cells at the single cell level reveals high similarities between rodents and humans. Pain 10.1097/j.pain.0000000000002628 (Epub Mar. 31, 2022)). RNAscope in situ hybridization shows GPR37L1 mRNA expression in human SGCs co-expressing glutamate synthase (GS), a specific marker for SGCs (data not shown). Double IHC staining showed co-localization of GPR37L1 with FABP7, a SGC marker (data not shown; see also O. Avraham et al. 2022, Satellite glial cells promote regenerative growth in sensory neurons. Nat Commun 11(1), Article 4891). Intriguingly, GPR37L1 was enriched on the inner side of SGCs that are in close contact with neurons (data not shown), providing an anatomical substrate for GPR37L1 to mediate neuron-glial interaction.

### Example 3: Loss-of-Function and Gain-of-Function Studies in Mice

The baseline pain sensitivity (physiological pain) including heat sensitivity, cold sensitivity, and mechanical sensitivity in littermates of Gpr37l1^{+/+}, Gpr37l1^{+/-}, and Gpr37l1^{-/-} mice was examined. No changes in heat sensitivity (hot plate testing), cold sensitivity (acetone testing), and mechanical sensitivity (von Frey testing) between the littermate control and Gpr37l1 mutants were observed (data not shown; see U.S. Prov. No. 63/270,198, FIG. 2A-2C). Not intending to be bound by theory, Gpr37l1^{-/-} may not be required for the genesis of physiological pain under normal conditions.

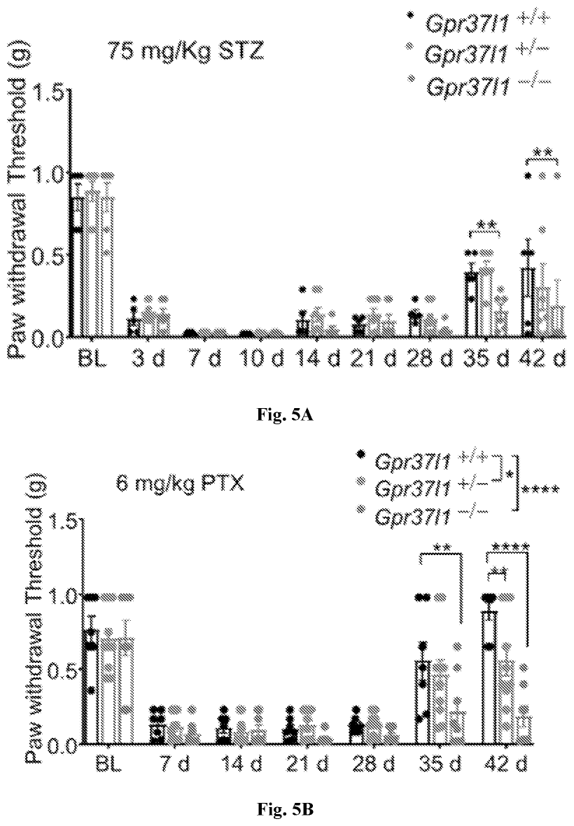

Two neuropathic pain models were created by systemic injection of streptozotocin (STZ), a diabetes-induced toxin, and paclitaxel (PTX), a chemotherapy drug, to see whether Gpr37L1 improves pain resolution. Gpr37^{-/-} mice had normal baseline (BL) pain before injury, compared to WT mice (FIGS. 5A-5B). The chemotherapy drug PTX (6 mg/kg, IP) induced mechanical allodynia 1-4 weeks after the injection, but this allodynia recovered after 5 weeks in WT mice. In Gpr37^{-/-} mice (“KO mice”), development of chemotherapy-induced neuropathic pain was not altered, compared to WT mice. Not intending to be bound by any particular theory, the lack of changes in baseline (BL) pain in KO mice could be a result of developmental compensation. However, the resolution of neuropathic pain at week 5 (35 d) and week 6 (42 d) after paclitaxel treatment is significantly impaired in KO mice (n=7-10 mice, Two-Way ANOVA with Tukey's posthoc test, *p<0.05, **p<0.01, ****p<0.0001. FIG. 5B). Not intending to be bound by theory, this result suggested that GPR37L1 may be required for the resolution of neuropathic pain.

The time course of STZ and PTX induced mechanical allodynia was tested in Gpr37l1^{+/+}, Gpr37l1^{+/-}, and Gpr37l1^{-/-} mice. STZ evoked rapid mechanical allodynia in 3 days, which began to resolve on Day 35 (FIG. 5A). Interestingly, Gpr37l1^{-/-} mice showed no sign of resolution on Day 35 and Day 42, compared to Gpr37l1^{+/+} mice (p<0.01 on Day 35, p<0.05 on Day 42, FIG. 5A). PTX also elicited profound mechanical allodynia in 7 days, which began to resolve on Day 35 and fully resolved on Day 42 (FIG. 5B). Notably, Gpr37l1^{-/-} mice showed no sign of resolution on Day 35 and Day 42, compared to Gpr37l1^{+/+} mice (p<0.01 on Day 35, p<0.0001 on Day 42, FIG. 5B). Not intending to be bound by theory, the results suggest that GPR37L1 may be required for the resolution of diabetes and chemotherapy induced neuropathic pain.

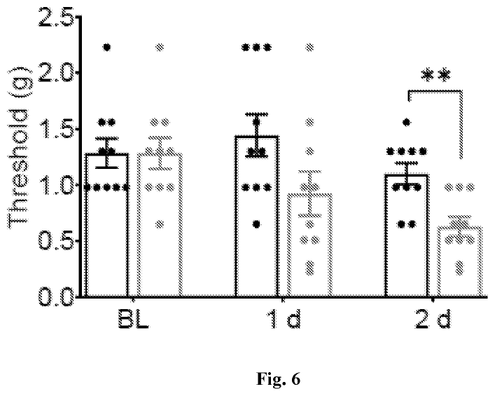

To further determine whether loss of GPR37L1 in I animal scan cause pain, a knockdown experiment was conducted using specific siRNA that targets Gpr37l1 expression. Unilateral intraganglionic microinjection of 2 μL of Gpr37l1-targeting siRNA (4 μg) in the L4 and L5 DRGs reduced Gpr37l1 expression by 50% and induced persistent mechanical allodynia in naïve animals for >2 days as shown in FIG. 6 (**p<0.01, siRNA (right bars) vs. scrambled control RNA, scRNA (left bars), n=10 mice). Not intending to be bound by theory, this result suggests that a partial loss of GPR37L1 is sufficient to produce a pain state in naïve animals.

### Example 4: Gain-of-Function Studies in Mice

Over-expression of Gpr37l1 in DRGs may be sufficient to reverse/resolve chronic pain after CIPN and DPN. To test this, intraganglionic or intrathecal AAV5 or AAV9 virus injections can be introduced to over-express GPR37L1 in paclitaxel or STZ treated mice at 2 weeks after the induction of CIPN and DPN. It will take 2-4 weeks for the AAV expression in DRG. The time course of mechanical pain (von Frey test) and cold pain (acetone test) can be assessed. Ongoing pain (CPP) can also be tested at certain time points with intrathecal clonidine. (T. King et al., 2009, Unmasking the tonic-aversive state in neuropathic pain. Nat. Neurosci. 12(11):1364-1366). Whether CIPN and DPN induced neuroinflammation can be reversed by Gpr37l1 over-expression will also be examined.

To specifically target SGCs in the DRGs and astrocytes in the spinal cord, an Aldh1l1-Cre/ERT2; AAV [FLEXon]-Gpr37l1 virus will be locally injected to the DRG and spinal cord, followed by 5 daily Tamoxifen injections to induce the Cre-virus expression. Not intending to be bound by theory, results may define distinct roles of GPR37L1 at DRG and spinal cord levels. If targeting GPR37L1 at DRG level is sufficient to resolve pain, then the lead compounds (GPR37L1 agonist) do not need to penetrate the brain-blood barrier, which may minimize the CNS-related side effects.

### Example 5: MaR1 Binds GPR37L1

Neuroprotectin D1 (NPD1), or protectin D1 (PD1), has been recognized as a ligand for GPR37. To search for the ligands of GPR37L1, the specialized pro-resolving mediators (SPMs), including D-resolvins (RvD1, RvD2, RvD5), E-resolvin (RvE1), NPD1, MaR1, and their precursors DHA and EPA were tested. A lipid overlay assay was conducted to reveal any possible binding of these lipid mediators to GPR37L1. hGPR37L1 was FLAG-tagged and expressed in HEK293 cells after hGPR37L1 cDNA transfection; the expressed protein detectable by anti-FLAG antibody (schematic not shown; see U.S. Prov. No. 63/270,198, FIG. 3A). In the plate coated with different lipid mediators it was shown that only MaR1 showed a specific binding signal (data not shown; see U.S. Prov. No. 63/270,198, FIG. 3B). MaR1 showed no positive signal in cell lysate from control mock transfection (data not shown; see U.S. Prov. No. 63/270,198, FIG. 3B).

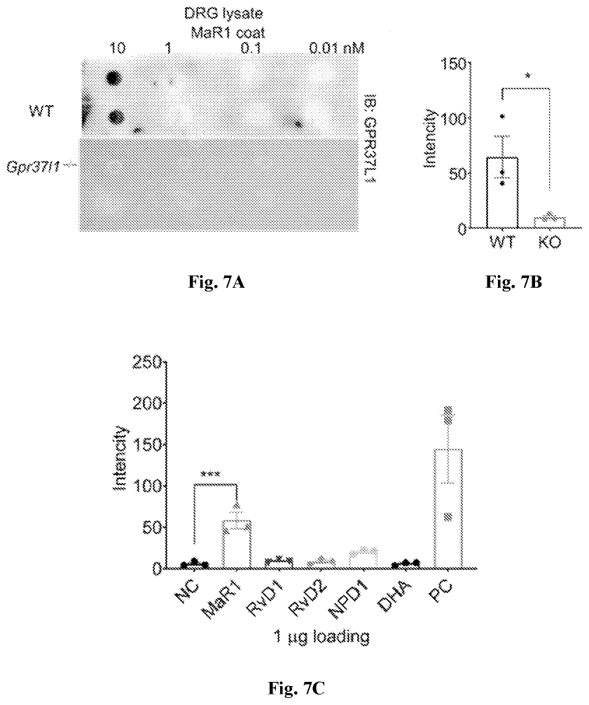

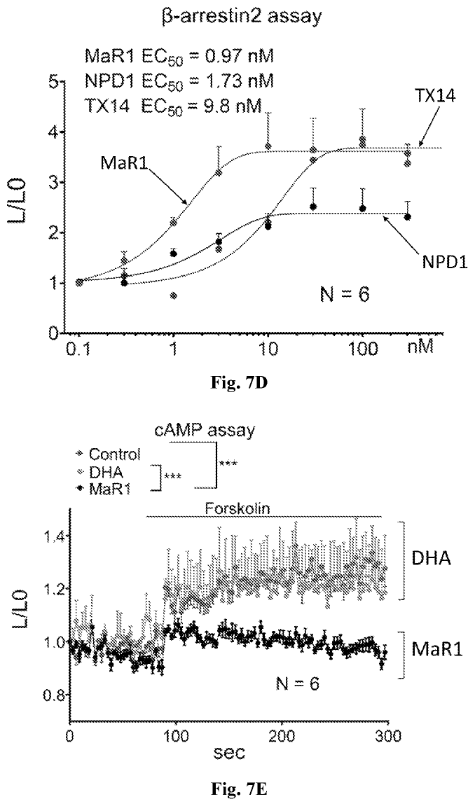

To confirm whether DRG-expressed GPR37L1 would also interact with MaR1, a lipid overlay assay was performed on MaR1-coated PVDF membrane using DRG lysates from WT or Gpr37l1 KO mice. To determine dose-dependent binding, MaR1 was coated at the concentrations of 0.01 nM, 0.1 nM, 1 nM, and 10 nM. A specific binding spot was observed at 10 nM MaR1 in WT DRG lysate. But this binding was completely absent in KO DRG lysate (p<0.05, n=3, FIGS. 7A-7B), confirming that this binding is GPR37L1-specific. These data suggest that MaR1 may bind to GPR37L1 in the mouse DRG.

Next, a lipid-coated bead pull-down assay with hGPR37L1-expressing cells was used to confirm whether MaR1 would interact with GPR37L1 protein selectively. Beads were coated with MaR1, RvD1, RvD2, NPD1, and their precursor DHA. Strong GPR37L1 binding in MaR1-coated bead pull-down extracts was observed and some weak binding in NPD1-coated bead pull-down extracts, but not binding to RvD1, RvD2, and DHA (data not shown; see U.S. Prov. No. 63/270,198, FIG. 3E). The right molecule size of GPR37L1 (˜50 kDa) was also validated by the anti-FLAG antibody (data not shown; see U.S. Prov. No. 63/270,198, FIG. 3E). In contrast, protein lysate from Mock transfection showed no binding (FIG. 7C). Quantitative analysis indicated that MaR1 binding intensity was significantly higher than the mock control/normal control (NC, p<0.01, n=3, FIG. 7C). The results suggested a direct binding of MaR1 to GPR37L1.

The Path Hunter hGPR37L1 ChoK1 system (DiscoverX) was used to test GPR37L1-mediated β-arrestin activation in response to MaR1, NPD1, and TX14, a potential ligand for GPR37 and GPR37L1 (R. C. Meyer, et al., GPR37 and GPR37L1 are receptors for the neuroprotective and glioprotective factors prosaptide and prosaposin. Proc Natl Acad Sci USA 110, 9529-9534 (2013)) using the DiscoverX cells expressing hGPR37L1. Eight different concentrations of compounds ranging from 0.1 nM to 100 nM were tested. MaR1 induced a potent β-arrestin signaling (3.64 fold increase), with an EC₅₀=0.97 nM (FIG. 7D; n=6). TX14 also elicited a strong β-arrestin response at higher concentrations (3.71 fold increase) with an EC₅₀=9.8 nM. NPD1 evoked a weak β-arrestin response (1.8 fold increase), with an EC₅₀=1.74 nM (FIG. 7D; n=6). Furthermore, the cAMP-BRET assay showed that MaR1 (10 nM), but not its precursor DHA (100 nM), significantly inhibited forskolin-induced cAMP production in GPR37L1 and cAMP BRET co-transfected cells (FIG. 7E; n=3; p<0.001 vs control). Not intending to be bound by theory, data suggests that 1) MaR1 is a potent ligand for GPR37L1 and 2) GPR37L1 appears to be Gi-coupled following MaR1 stimulation.

### Example 6: Computer Simulations Reveal MaR1 Interaction with GPR37L1 at Specific Sites

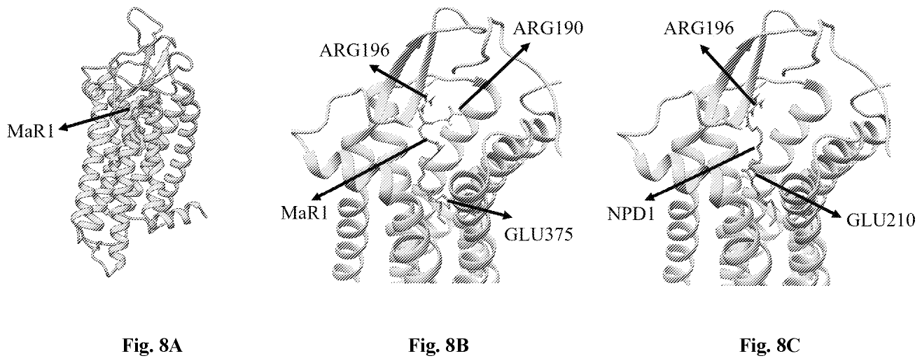

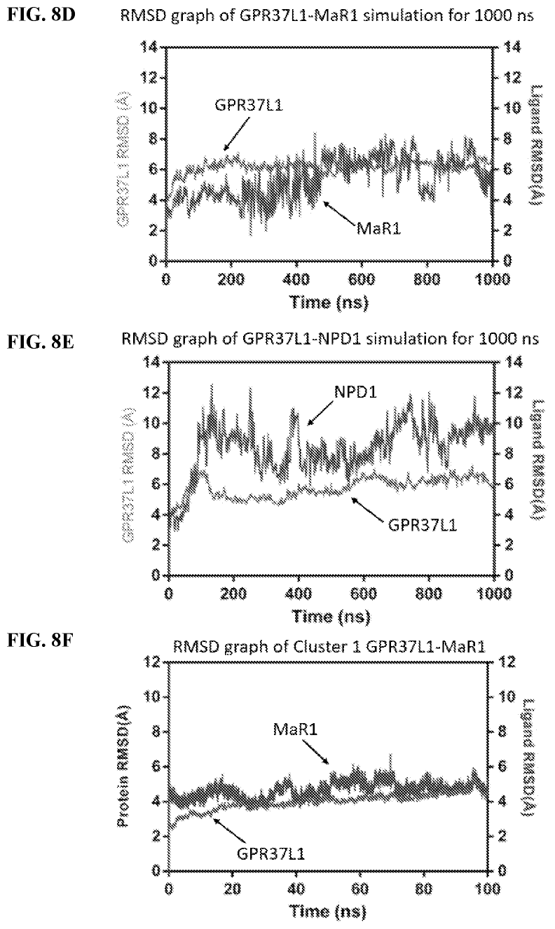

Computational prediction of a potential interaction between MaR1 and GPR37L1 was performed using GPR37L1 homology modeling. The human endothelial receptor B (human ETB sequence) was used for GPCR transmembrane helix sequence alignment with human GPR37L1 (schematic not shown; see U.S. Prov. No. 63/270,198, FIG. 4A). The crystal structure of Human ETB (PDB: 6IGK) was used as template for the GPR37L1 structure modeling (schematic not shown; see U.S. Prov. No. 63/270,198, FIG. 4B). The structures of MaR1 and NPD1 (see Table 1; see also U.S. Prov. No. 63/270,198, FIG. 4C) and their interactions with GPR37L1 were shown (FIGS. 8A-8C). It was determined that MaR1 interacts with GPR37L1 by assessing the hydrogen bonding strength using molecular docking and molecular dynamic simulation (MDS) (FIGS. 8A-8C). MaR1 forms hydrogen bonding with ASN190, ARG196, and GLU375 residues in the GPR37L1 binding pocket. However, NPD1 binds within the same pocket and forms hydrogen bond with ARG196 and GLU210. In RMSD calculations of 1000 ns simulation, the MaR1-GPR37L1 complex showed stable binding (FIG. 8B). In contrast, NPD1 binds in the same pocket, but without interactions with ARG196 and GLU375 in combination (FIG. 8C). The NPD1-GPR37L1 complex showed less stable interaction (FIG. 8E). From the 100 ns MDS of GPR37L1-MaR1 complex cluster 1, RMSD value of the protein backbone was stabilized at 4 Å and the RMSD value of MaR1 was between 4 to 6 Å (FIG. 8F). Extensive computational approaches including molecular docking of MaR1 were also used on binding pocket of GPR37L1, a deep neural network-based algorithm for the prediction of binding energy. This simulation approach resulted in the identification of 16 novel small-molecule GPR37L1 binders that have lower binding energy than MaR1 with lower ΔG values (Table 1). As expected, MaR1 has a lower binding energy than NPD1 (ΔG value: −9.12127 for MaR1; −8.75108 for NPD1) (Table 1).

TABLE 1
Identified chemical compounds by AI-based docking simulation of GPR37L1 and their ΔG values.

| | Compound Name | | ΔG |
|---|---|---|---|
| 1 | MaR1 | 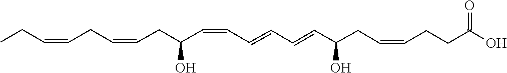 [SMILES: CC/C=C\C/C=C\C[C@H](O)\C=C/C=C/C=C/[C@H](O)C/C=C\CCC(=O)O] | −9.12127 |
| 2 | NPD1 | 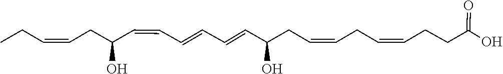 [SMILES: CC/C=C\C[C@H](O)\C=C/C=C/C=C/[C@H](O)C/C=C\C/C=C\CCC(=O)O] | −8.75108 |
| 3 | MCULE-6965498156 CAS: 130209-76-6 | 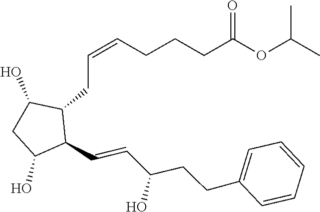 [SMILES: CC(C)OC(=O)CCC/C=C\C[C@@H]1[C@@H](/C=C/[C@@H](O)CCc2ccccc2)[C@H](O)C[C@@H]1O] | −10.3432 |
| 4 | MCULE-3117827100 CAS: 931223-08-4 | 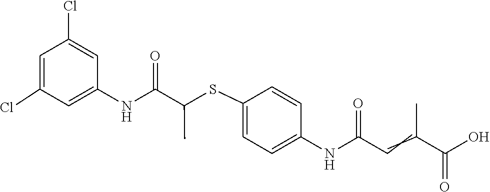 [SMILES: CC(=CC(=O)Nc1ccc(SC(C)C(=O)Nc2cc(Cl)cc(Cl)c2)cc1)C(=O)O] | −10.1356 |
| 5 | MCULE-5609766020 CAS: 59685-93-7 | 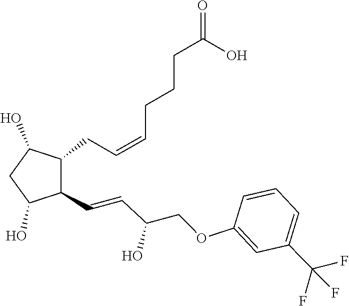 [SMILES: O=C(O)CCC/C=C\C[C@@H]1[C@@H](/C=C/[C@@H](O)COc2cccc(C(F)(F)F)c2)[C@H](O)C[C@@H]1O] | −10.0334 |
| 6 | MCULE-2884027413 (P06) | 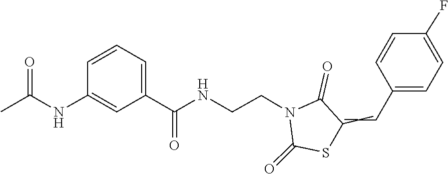 [SMILES: CC(=O)Nc1cccc(C(=O)NCCN2C(=O)SC(=Cc3ccc(F)cc3)C2=O)c1] | −9.94278 |
| 7 | MCULE-1786116136 CAS: 157283-66-4 | 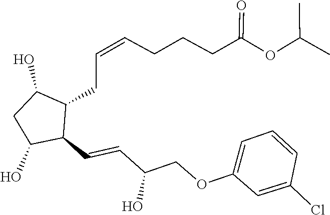 [SMILES: CC(C)OC(=O)CCC/C=C\C[C@@H]1[C@@H](/C=C/[C@@H](O)COc2cccc(Cl)c2)[C@H](O)C[C@@H]1O] | −9.94244 |
| 8 | MCULE-2175470034 CAS: 1094995-75-1 | 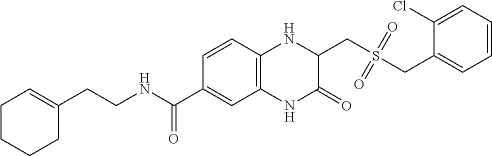 [SMILES: O=C(NCCC1=CCCCC1)c1ccc2c(c1)NC(=O)C(CS(=O)(=O)Cc1ccccc1Cl)N2] | −9.91784 |
| 9 | MCULE-9605880790 CAS: 875343-22-9 | 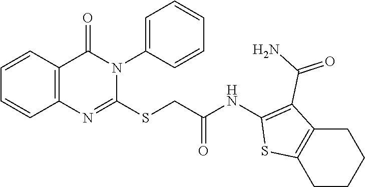 [SMILES: NC(=O)c1c(NC(=O)CSc2nc3ccccc3c(=O)n2-c2ccccc2)sc2c1CCCC2] | −9.87447 |
| 10 | MCULE-2709239856 | 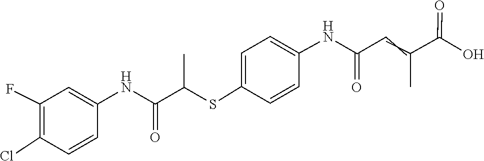 [SMILES: CC(=CC(=O)Nc1ccc(SC(C)C(=O)Nc2ccc(Cl)c(F)c2)cc1)C(=O)O] | −9.85704 |
| 11 | MCULE-1615794560 | 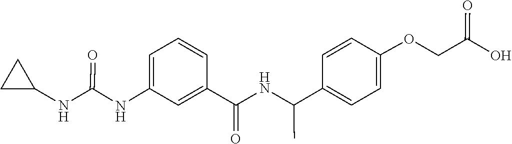 [SMILES: CC(NC(=O)c1cccc(NC(=O)NC2CC2)c1)c1ccc(OCC(=O)O)cc1] | −9.83801 |
| 12 | MCULE-1784164690 | 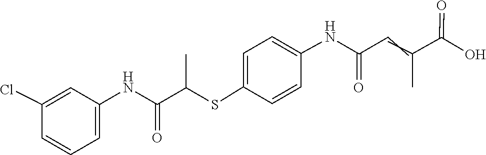 [SMILES: CC(=CC(=O)Nc1ccc(SC(C)C(=O)Nc2cccc(Cl)c2)cc1)C(=O)O] | −9.82495 |
| 13 | MCULE-7360829293 | 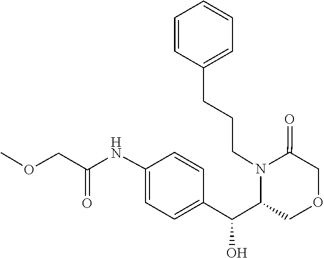 [SMILES: COCC(=O)Nc1ccc([C@@H](O)[C@H]2COCC(=O)N2CCCc2ccccc2)cc1] | −9.80105 |
| 14 | MCULE-2030135661 CAS: 1379782-00-9 | 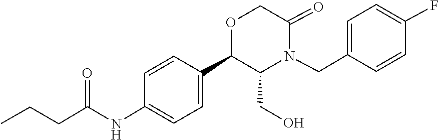 [SMILES: CCCC(=O)Nc1ccc([C@H]2OCC(=O)N(Cc3ccc(F)cc3)[C@@H]2CO)cc1] | −9.78863 |
| 15 | MCULE-2500192016 CAS: 919920-00-6 | 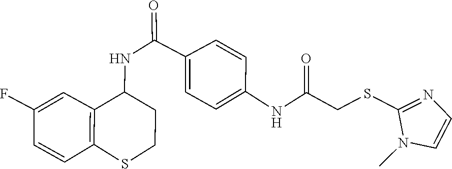 [SMILES: Cn1ccnc1SCC(=O)Nc1ccc(C(=O)NC2CCSc3ccc(F)cc32)cc1] | −9.75447 |
| 16 | MCULE-2227574192 CAS: 950096-26-1 | 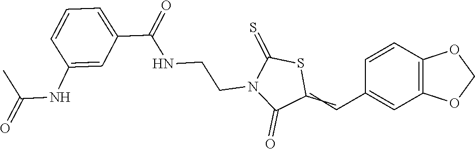 [SMILES: CC(=O)Nc1cccc(C(=O)NCCN2C(=O)C(=Cc3ccc4c(c3)OCO4)SC2=S)c1] | −9.7221 |
| 17 | MCULE-5725483684 CAS: 919631-74-6 | 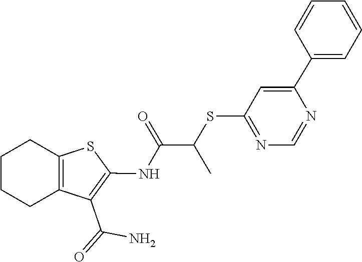 [SMILES: CC(Sc1cc(-c2ccccc2)ncn1)C(=O)Nc1sc2c(c1C(N)=O)CCCC2] | −9.65572 |
| 18 | MCULE-8222044844 CAS: 2038048-58-5 | 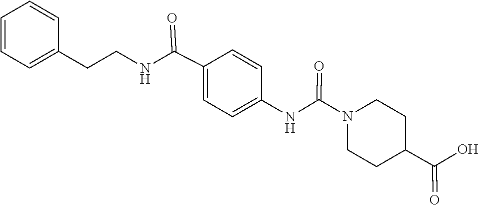 [SMILES: O=C(NCCc1ccccc1)c1ccc(NC(=O)N2CCC(C(=O)O)CC2)cc1] | −9.62815 |

### Example 7: MaR1 Inhibits Neuropathic Pain Via GPR37L1

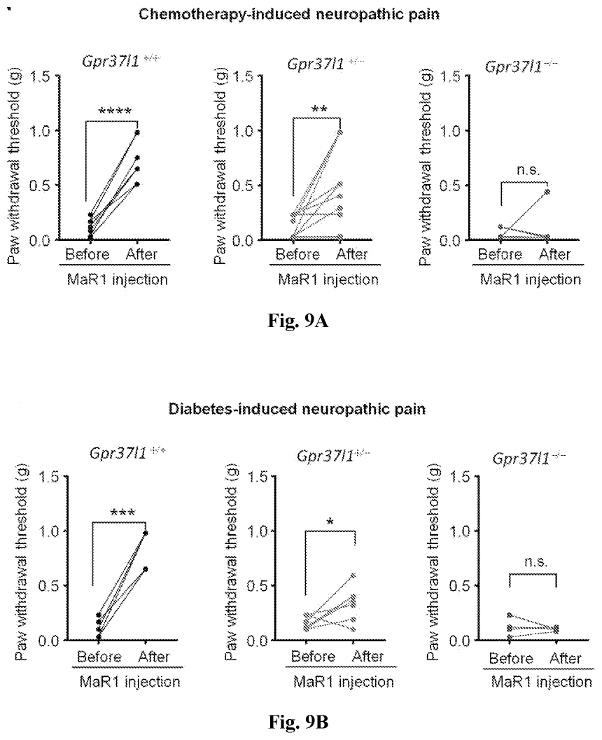

The analgesic actions of MaR1 in chemotherapy and diabetes-induced neuropathic pain were tested in Gpr37l1^{+/+}, Gpr37l1^{+/-}, and Gpr37l1^{-/-} mice, following intrathecal injection (100 ng) to target DRG cells including SGCs, as well as spinal cord cells. In Gpr37l1^{+/+} WT mice, MaR1 reversed PTX-induced mechanical allodynia in all the mice, with highly significant difference (FIG. 9A, p<0.0001, n=7 mice). MaR1 also significantly reversed the mechanical allodynia in a majority of Gpr37l1^{+/-} mice (FIG. 9A, p<0.01, n=10 mice). By contrast, MaR1 had no significant effects on Gpr37l1^{-/-} mice (FIG. 9A, p>0.05, n=8 mice). Similar effects were also observed on MaR1 in the STZ model (FIG. 9B). In Gpr37l1^{+/+} WT mice, MaR1 reversed STZ-induced mechanical allodynia in all the mice, with highly significant difference (FIG. 9B, p<0.001, n=5 mice). MaR1 also significantly reversed the mechanical allodynia in the majority of Gpr37l1^{+/-} mice (FIG. 9B, p<0.05, n=7 mice). By contrast, MaR1 had no significant effects on mechanical allodynia in Gpr37l1^{-/-} mice (FIG. 9B, p>0.05, n=6 mice). Not intending to be bound by theory, data suggests that 1) MaR1 is highly effective in attenuating neuropathic pain in both models and 2) GPR37L1 is essential for MaR1 to reduce neuropathic pain.

### Example 8: MaR1 Regulates IL-1β Release and Potassium Currents in SGCs Via GPR37L1

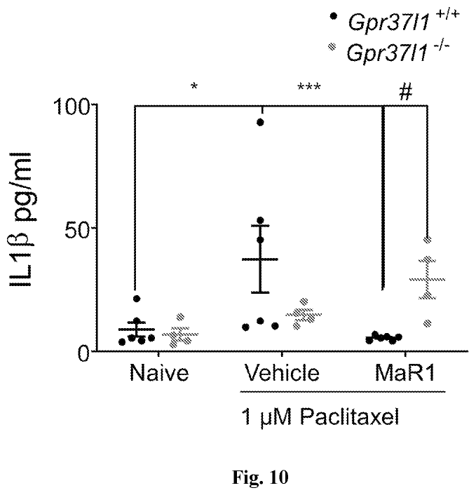

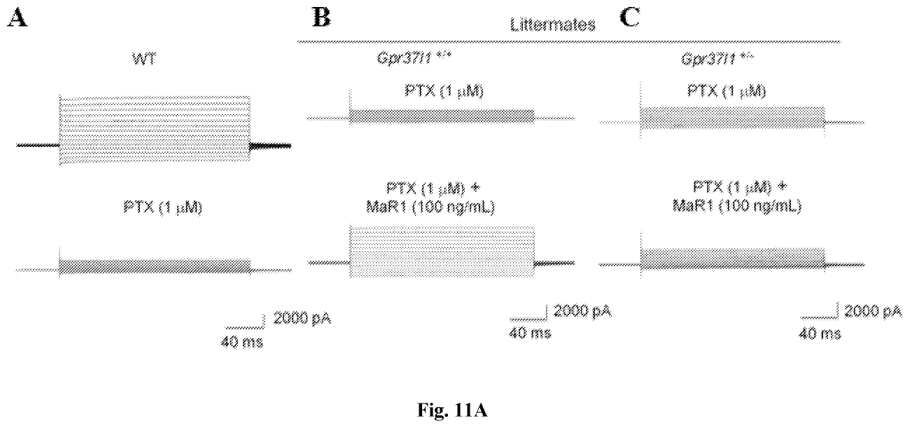

Following painful insults, SGCs release pro-inflammatory cytokines, such as IL-1β to activate nociceptive sensory neurons (Y. Kawasaki et al., Distinct roles of matrix metalloproteases in the early- and late-phase development of neuropathic pain. Nat. Med. 14, 331-336 (2008); A. M. Binshtok et al., Nociceptors are interleukin-1beta sensors. J. Neurosci. 28, 14062-14073 (2008)). To examine SGC-mediated neuro-glial interactions in pathological conditions and the involvement of GPR37L1, neuron-glia mixed cultures were prepared from DRG of WT and Gpr37l1^{-/-} mice. The co-culture was stimulated with 1 μM paclitaxel for 24 h. ELISA result showed a marked increase in IL-1β secretion in culture medium, and this increase was blocked by 100 nM MaR1 in cells from WT but not KO mice (FIG. 10). Not intending to be bound by theory, data suggests that MaR1 can inhibit chemotherapy-induced IL-1β release via GPR37L1.

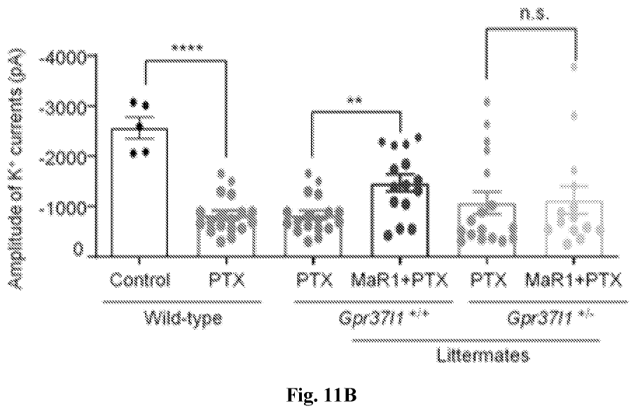

Kir4.1 potassium channels are the predominant potassium channels in SGCs and are chiefly responsible for the potassium currents in SGCs. Dysregulation of SGC Kir4.1 contributes to the pathogenesis of pain (M. Hanani, D. C. Spray, Emerging importance of satellite glia in nervous system function and dysfunction. Nat Rev Neurosci 21, 485-498 (2020)). SGC K⁺ currents were recorded by conducting patch-clamp recordings in SGCs in whole-mount DRG preparation (data not shown; see U.S. Prov. No. 63/270,198, FIG. 7A), collected from WT mice, Gpr37l1^{+/+} mice, and Gpr37l1^{+/-} mutant mice (FIG. 11A parts A-C). SGCs in WT-DRG exhibited large K⁺ currents (Iₖ₋₁₆₀, >2200 pA) when the holding potential was −160 mV (FIG. 11A part A). The total currents were largely blocked by the Kir4.1 channel blocker Barium (100 μM) (see X. Tong et al., Astrocyte Kir4.1 ion channel deficits contribute to neuronal dysfunction in Huntington's disease model mice. Nat Neurosci 17, 694-703 (2014)). Thus, the amplitude of the Kir4.1-mediated Iₖ₋₁₆₀ current was comparable with that of the total K⁺ currents. PTX treatment (1 μM, 60 min) produced a substantial reduction in the K⁺ currents (p<0.0001, FIG. 11A part A and FIG. 11B). Intriguingly, co-application of MaR1 (100 ng/ml) with PTX significantly rescued the PTX-induced downregulation of the K⁺ currents (p<0.01, FIG. 11A part B and FIG. 11B). However, the MaR1's effects on K⁺ currents were completely abolished in Gpr37l1 mutant (FIG. 11A part C and FIG. 11B). Not intending to be bound by theory, these data suggest 1) chemotherapy could potently down-regulated Kir4.1 activity in SGCs and 2) MaR1 is able to confer protection against this downregulation via GPR37L1.

What is claimed is:

1. A method of treating pain in a subject, the method comprising administering a therapeutically effective amount of a GPR37L1 ligand to the subject.

2. The method of claim 1, wherein the GPR37L1 ligand is selected from the group consisting of pro-resolving lipid mediator maresin 1 (MaR1), NPD1, MCULE-6965498156, MCULE-3117827100, MCULE-5609766020, MCULE-2884027413, MCULE-1786116136, MCULE-2175470034, MCULE-9605880790, MCULE-270239856, MCULE-1615794560, MCULE-1784164690, MCULE-7360829293, MCULE-2030135661, MCULE-2500192016. MCULE-2227574192, MCULE-5725483684, MCULE-8222044844, any fragments, derivatives, salts, esters, and variants thereof, and any combinations thereof.

3. The method of claim 1, wherein the method comprises administering pro-resolving lipid mediator maresin 1 (MaR1), or a fragment, derivative, salt, ester, or variant thereof to the subject.

4. The method of claim 1, wherein the pain comprises inflammatory pain, cancer pain, or neuropathic pain.

5. The method of claim 1, wherein the pain is mechanical allodynia.

6. The method of claim 1, wherein the pain is at least one of headache or low-back pain.

7. The method of claim 1, wherein the GPR37L1 ligand is administered intrathecally or intraperitoneally.

8. The method of claim 1, wherein the GPR37L1 ligand has the structure of:

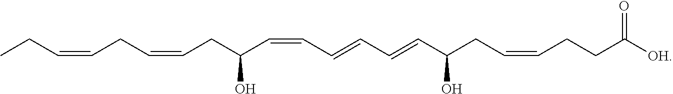 [SMILES: CC/C=C\C/C=C\C[C@H](O)\C=C/C=C/C=C/[C@H](O)C/C=C\CCC(=O)O]

9. The method of claim 1, wherein the GPR37L1 ligand has the structure of:

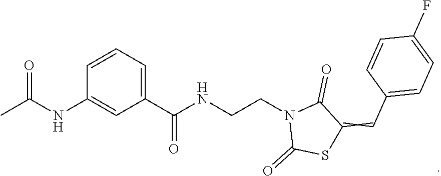

10. The method of claim 1, wherein the subject is undergoing treatment for cancer.

11. The method of claim 10, wherein the subject is undergoing a chemotherapeutic regimen.

12. The method of claim 11, wherein the chemotherapeutic regimen comprises administration of paclitaxel or oxaliplatin.

13. The method of claim 10, wherein the subject is being administered radiation therapy.

14. A pharmaceutical composition comprising a GPR37L1 ligand and a pharmaceutically acceptable diluent, excipient, and/or carrier.

15. The pharmaceutical composition of claim 14, wherein the GPR37L1 ligand is selected from the group consisting of pro-resolving lipid mediator maresin 1 (MaR1), NPD1, MCULE-6965498156, MCULE-3117827100, MCULE-5609766020, MCULE-2884027413, MCULE-1786116136, MCULE-2175470034, MCULE-9605880790, MCULE-270239856, MCULE-1615794560, MCULE-1784164690, MCULE-7360829293, MCULE-2030135661, MCULE-2500192016. MCULE-2227574192, MCULE-5725483684, MCULE-8222044844, any fragments, derivatives, salts, esters, and variants thereof and combinations thereof.
